SEQUENCE LISTING (6 sequences)

SEQ ID NO: 1 - protein, 269 aa, Bacillus clausii
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 2 - protein, 275 aa, Bacillus amyloliquefaciens [sequence omitted]

SEQ ID NO: 3 - protein, 269 aa, Artificial Sequence
  Variant of SEQ ID NO1
AQTIPWGISRVQAPAAHNRGLTGSGVKVAVLDTGISTHPDLNIRGGASFVPGEPSTQDGNGHGTHVAGTIAALNNSIGVLGVAPSAELYAVKVLGADGEGAISSIAQGLEWAGNNGMHVANLSLGSPSPSATLEQAVNSATSRGVLVVAASGNSGASSISYPARYANAMAVGATDQNNNRASFSQYGAGLDIVAPGVNIQSTYPGSTYASLNGTSMATPHVAGAAALVKQKNPSWSNVQIRNHLKNTATSLGSTNLYGSGLVNAEAATR

SEQ ID NO: 4 - protein, 270 aa, Artificial Sequence
  Variant of SEQ ID NO1
AQSVPWGISRVQAPAAHNRGLTGSGVKVAVLDTGIDSTHPDLNIRGGASFVPGEPSTQDGNGHGTHVAGTIAALDNSIGVLGVAPSAELYAVKVLGASGSGSVSSIAQGLEWAGNNGMDVANLSLGSPSPSATLEQAVNSATSRGVLVVAASGNSGAGSISYPARYANAMAVGATDQNNNRASFSQYGAELDIVAPGVNVQSTYPGSTYASLNGTSMATPHVAGAAALVLQKNPSWSNVQIRNHLKNTATSLGSTNLYGSGLVNAEAATR

SEQ ID NO: 5 - protein, 269 aa, Artificial Sequence
  Variant of SEQ ID NO1
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSISYPARYANAMAVGATDQNNNRASFSQYGAGLDIVAPGVNILSTWPGSTYASLNGTSMATPHVAGAAALVKQKNPSWSNVQIRNHLKNTATSLGDTWEYGSGLVNAEAATR

SEQ ID NO: 6 - protein, 269 aa, Artificial Sequence
  Variant of SEQ ID NO1
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNILSTWPGSTYASLNGTSMATPHVAGAAALVKQKNPSWSNVQIRNHLKNTATSLGDTWEYGSGLVNAEAATR

(19) United States
(12) Patent Application Publication
(10) Pub. No.: US 2023/0265408 A1
(43) Pub. Date: Aug. 24, 2023

(54) PROTEASE VARIANTS WITH IMPROVED SOLUBILITY

(71) Applicant: Novozymes A/S, Bagsvaerd (DK)

(72) Inventors: Rolf Thomas Lenhard, Lyngby (DK); Esben Peter Friis, Herlev (DK); Poul Erik Pedersen, Farum (DK); Rasmus Lund Andersen, Vintrie (SE)

(73) Assignee: Novozymes A/S, Bagsvaerd (DK)

(21) Appl. No.: 18/043,532

(22) PCT Filed: Aug. 30, 2021
(86) PCT No.: PCT/EP2021/073869

§ 371 Date: Feb. 28, 2023

(30) Foreign Application Priority Data
Aug. 28, 2020 (EP) 20193301.7
Feb. 12, 2021 (EP) 21156744.1

(51) Int. Cl. C12N 9/50; C12N 15/63; C11D 3/386

(52) U.S. Cl. CPC C12N 9/50; C12N 15/63; C11D 3/386

(57) ABSTRACT

The present invention relates to protease variants, polynucleotides encoding said variants, nucleic acid constructs and expression vectors comprising said polynucleotides, host cells expressing said variants, methods of obtaining the variants, detergent compositions comprising said variants, and use of said variants or said detergent compositions.

### REFERENCE TO A SEQUENCE LISTING

This application contains a Sequence Listing in computer readable form, which is incorporated herein by reference.

### FIELD OF THE INVENTION

The present invention relates to protease variants, polynucleotides encoding said variants, nucleic acid constructs and expression vectors comprising said polynucleotides, host cells expressing said variants, methods of obtaining the variants, detergent compositions comprising said variants, and use of said variants or said detergent compositions.

### BACKGROUND OF THE INVENTION

In the detergent industry, enzymes have been implemented in washing formulations for many decades. Enzymes used in such formulations include proteases, lipases, amylases, cellulases, mannosidases as well as other enzymes or mixtures thereof. Commercially, the most important enzymes are proteases.

An increasing number of commercially used proteases for, e.g., laundry and dishwashing detergents are protein engineered variants of naturally occurring wild type proteases. Further, other protease variants have been described in the art with alterations relative to a parent protease resulting in improvements such as better wash performance, thermal stability, storage stability or catalytic activity.

However, various factors make further improvement of proteases advantageous. For example, washing conditions such as temperature and pH tend to change over time, and are also different in different countries or regions of the world, and many stains are still difficult to completely remove under conventional washing conditions.

Another challenge relating to proteases is their solubility. The solubility of proteases is an important factor when producing these enzymes since proteases of low solubility are more likely to crystallize during fermentation and downstream processing. A protease with high solubility can be processed at higher concentrations, making the process of purifying the protease cheaper, faster and more sustainable.”

The present invention addresses this challenge by providing protease variants with improved solubility.

### SUMMARY OF THE INVENTION

The present invention provides protease variants with improved solubility. The protease variants of the invention comprise a positively charged or polar amino acid at a position corresponding to position 215 of SEQ ID NO:1.

Thus, in a first aspect, the present invention relates to a protease variant of a parent protease, wherein the variant has a sequence identity of at least at least 80%, but less than 100%, to SEQ ID NO:1;
wherein the variant comprises a first substitution selected from the group consisting of X215K, X215R, X215Q, X125N, X215S, and X215T;

wherein the variant comprises at least three further alterations, preferably substitutions, selected from the group consisting of X3T (e.g., S3T), X4I (e.g., V4I), X9E (e.g., S9E), I35ID, X43R (e.g., N43R), X76D (e.g., N76D), X99D (e.g., S99D, X99F (e.g., S99F), X101E (e.g., S101E), X101L (e.g., S101L), X103A (e.g., S103A), X103T (e.g., S103T), X104I (e.g., V104I), X120D (e.g., H120D), X160S (e.g., G160S), X195E (e.g., G195E), X205I (e.g., V205I), X206L (e.g., Q206L), X209W (e.g., Y209W), X235L (e.g., K235L), X259D (e.g., S259D), X261W (e.g., N261W), and X262E (e.g., L262E);

wherein the variant has protease activity; and

wherein position numbers are based on the numbering of SEQ ID NO:2.

In a second aspect, the present invention relates to a polynucleotide encoding a protease variant of the first aspect.

In a third aspect, the present invention relates to a nucleic acid construct or expression vector comprising a polynucleotide of the second aspect.

In a fourth aspect, the present invention relates to a host cell expressing a protease variant according to the first aspect.

In a fifth aspect, the present invention relates to a method for obtaining a protease variant according to any of claims 1-15, the method comprising:

(a) introducing into a parent protease a first substitution selected from the group consisting of X215K, X215R, X215Q, X125N, X215S, and X215T; and introducing at least three further alterations, preferably substitutions, selected from the group consisting of X3T (e.g., S3T), X4I (e.g., V4I), X9E (e.g., S9E), I35ID, X43R (e.g., N43R), X76D (e.g., N76D), X99D (e.g., S99D, X99F (e.g., S99F), X101E (e.g., S101E), X101L (e.g., S101L), X103A (e.g., S103A), X103T (e.g., S103T), X104I (e.g., V104I), X120D (e.g., H120D), X160S (e.g., G160S), X195E (e.g., G195E), X205I (e.g., V205I), X206L (e.g., Q206L), X209W (e.g., Y209W), X235L (e.g., K235L), X259D (e.g., S259D), X261W (e.g., N261W), and X262E (e.g., L262E); wherein the variant has protease activity; and

(b) recovering the variant.

In a sixth aspect, the present invention relates to a detergent composition comprising a protease variant according to the first aspect.

In a seventh aspect, the present invention relates to use of a protease variant according to the first aspect or a detergent composition according to the sixth aspect in a cleaning process, preferably laundry or hard surface cleaning such as automated dish washing (ADW).

### BRIEF DESCRIPTION OF THE FIGURES

FIG. 1 shows an alignment between SEQ ID NO:1 and SEQ ID NO:2, based on Table 1 of WO 1989/06279, from which position numbers corresponding to positions of SEQ ID NO:2 may be readily determined.

### DEFINITIONS

Protease: The term “protease” means an enzyme that hydrolyses peptide bonds. It includes any enzyme belonging to the EC 3.4 enzyme group (including each of the thirteen subclasses thereof (http://en.wikipedia.org/wiki/Category:EC_3.4). The EC number refers to Enzyme Nomenclature 1992 from NC-IUBMB, Academic Press, San Diego, Calif., including supplements 1-5 published in Eur. J. Biochem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively. The term “subtilases” refer to a sub-group of serine protease according to Siezen et al., Protein Eng. 4 (1991) 719-737 and Siezen et al. Protein Science 6 (1997) 501-523. Serine proteases or serine peptidases is a subgroup of proteases characterized by having a serine in the active site, which forms a covalent adduct with the substrate. Further, the subtilases (and the serine proteases) are characterized by having two active site amino acid residues apart from the serine, namely a histidine and an aspartic acid residue. The subtilases may be divided into 6 sub-divisions, i.e. the Subtilisin family, the Thermitase family, the Proteinase K family, the Lantibiotic peptidase family, the Kexin family and the Pyrolysin family. The term “protease activity” means a proteolytic activity (EC 3.4). Protease variants of the invention are endopeptidases (EC 3.4.21). For purposes of the present invention, protease activity is determined according to the protease activity assay described in the Examples below.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a variant. The boundaries of the coding sequence are generally determined by an open reading frame, which begins with a start codon such as ATG, GTG or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a variant of the present invention. Each control sequence may be native (i.e., from the same gene) or foreign (i.e., from a different gene) to the polynucleotide encoding the variant or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a variant.

Expression: The term “expression” includes any step involved in the production of a variant including, but not limited to, transcription, post-transcriptional modification, translation, post-translational modification, and secretion.

Expression vector: The term “expression vector” means a linear or circular DNA molecule that comprises a polynucleotide encoding a variant and is operably linked to control sequences that provide for its expression.

Fragment: The term “fragment” means a polypeptide having one or more (e.g., several) amino acids absent from the amino and/or carboxyl terminus of a mature polypeptide; wherein the fragment has protease activity.

Fusion polypeptide: The term “fusion polypeptide” is a polypeptide in which one polypeptide is fused at the N-terminus or the C-terminus of a variant of the present invention. A fusion polypeptide is produced by fusing a polynucleotide encoding another polypeptide to a polynucleotide of the present invention. Techniques for producing fusion polypeptides are known in the art and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779). A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Hybrid polypeptide: The term “hybrid polypeptide” means a polypeptide comprising domains from two or more polypeptides, e.g., a binding module from one polypeptide and a catalytic domain from another polypeptide. The domains may be fused at the N-terminus or the C-terminus.

Improved property: The term “improved property” means a characteristic associated with a variant that is improved compared to the parent. Such improved properties include, but are not limited to, catalytic efficiency, catalytic rate, chemical stability, oxidation stability, pH activity, pH stability, polyester degrading activity, polyester specificity, proteolytic stability, solubility, specific activity, stability under storage conditions, substrate binding, substrate cleavage, substrate specificity, substrate stability, surface properties, thermal activity, and thermostability.

In one aspect, the variants of the invention have improved solubility. In particular, the variants of the invention exhibit decreased protease crystal formation, e.g., during fermentation, and/or increased protease crystal solubility (or, alternatively stated, improved protease crystal resolubilization). Protease crystal formation and protease crystal solubility may be determined according to the procedure described in Example 1 below. Protease crystal solubility may also be determined as the rate of protease crystal dissolution. Using this method, a protease is brought to crystallization by increasing its concentration (e.g., via a spin concentrator) in aqueous buffer and increasing the salt concentration and adjusting pH value until conditions suitable for crystallization are achieved. Following crystallization, protease crystal solubility may be determined by measuring the dissolution rate of the crystals.

In one aspect, the variants of the invention have on par or improved protease activity. Protease activity is determined according to the protease activity assay described in the Examples below.

Isolated: The term “isolated” means a polypeptide, nucleic acid, cell, or other specified material or component that is separated from at least one other material or component with which it is naturally associated as found in nature, including but not limited to, for example, other proteins, nucleic acids, cells, etc. An isolated polypeptide includes, but is not limited to, a culture broth containing the secreted polypeptide.

Mature polypeptide: The term “mature polypeptide” means a polypeptide in its mature form following N-terminal processing (e.g., removal of signal peptide).

Mutant: The term “mutant” means a polynucleotide encoding a variant.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Parent or parent protease: The term “parent” or “parent protease” means a protease to which an alteration is made to produce the enzyme variants of the present invention. The parent may be a naturally occurring (wild-type) polypeptide or a variant or fragment thereof.

Polymer: The term “polymer” means a chemical compound or mixture of compounds whose structure is constituted of multiple monomers (repeat units) linked by covalent chemical bonds. Within the context of the invention, the term polymer includes natural or synthetic polymers, constituted of a single type of repeat unit (i.e., homopolymers) or of a mixture of different repeat units (i.e., copolymers or heteropolymers). According to the invention, the term “oligomers”, when used in reference to a polymer, means molecules containing from 2 to about monomers.

Purified: The term “purified” means a nucleic acid or polypeptide that is substantially free from other components as determined by analytical techniques well known in the art (e.g., a purified polypeptide or nucleic acid may form a discrete band in an electrophoretic gel, chromatographic eluate, and/or a media subjected to density gradient centrifugation). A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

Recombinant: The term “recombinant,” when used in reference to a cell, nucleic acid, protein or vector, means that it has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding a polypeptide is a recombinant vector. The term “recombinant” is synonymous with “genetically modified” and “transgenic”.

Sequence identity: The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the -nobrief option must be specified in the command line. The output of Needle labeled “longest identity” is calculated as follows:

(Identical Residues×100)/(Length of Alignment−Total Number of Gaps in Alignment)

For purposes of the present invention, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows:

(Identical Deoxyribonucleotides×100)/(Length of Alignment−Total Number of Gaps in Alignment)

Variant and protease variant: The terms “variant” and “protease variant” means a polypeptide having protease activity comprising a substitution, an insertion, and/or a deletion, at one or more (e.g., several) positions compared to the parent. A substitution means replacement of the amino acid occupying a position with a different amino acid; a deletion means removal of the amino acid occupying a position; and an insertion means adding an amino acid adjacent to and immediately following the amino acid occupying a position. For purposes of the present invention, protease activity is determined according to the procedure described in the Examples below.

Wild-type: The term “wild-type” in reference to an amino acid sequence or nucleic acid sequence means that the amino acid sequence or nucleic acid sequence is a native or naturally occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild-type sequence).

### Conventions for Designation of Protease Variants

For purposes of the present invention, the polypeptide of SEQ ID NO:2 is used to determine the corresponding amino acid residue number in a variant of the invention. The amino acid sequence of a variant of the invention is aligned with SEQ ID NO:2, and based on the alignment, the amino acid position number corresponding to any amino acid residue in the variant of the invention.

The numbering used herein for SEQ ID NOs:1, 3, 4, 5, and 6 is based on the numbering of SEQ ID NO:2. Thus, for SEQ ID NOs:1, 3, 4, 5, and 6, the amino acid residues are numbered based on the corresponding amino acid residue in SEQ ID NO:2. Specifically, the numbering is based on the alignment in Table 1 of WO 1989/06279, which shows an alignment of five proteases, including the mature polypeptide of the subtilase BPN′ (BASBPN) sequence (sequence c in the table) and the mature polypeptide of subtilisin 309 from Bacillus clausii, also known as Savinase® (BLSAVI) (sequence a in the table). Persons skilled in the art will know that position numbers used for subtilisin 309 and other proteases in the patent literature are often based on the corresponding position numbers of BPN′ according to this alignment.

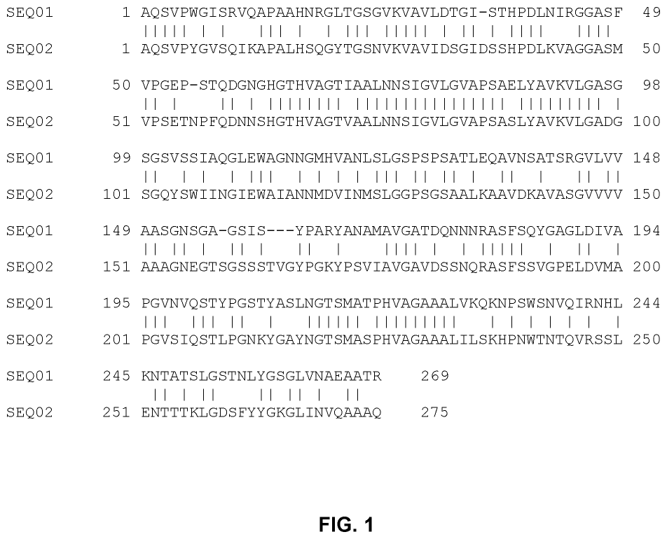

The accompanying FIG. 1 is provided for reference purposes and shows an alignment between SEQ ID NO:1 and SEQ ID NO:2, based on Table 1 of WO 1989/06279, from which position numbers corresponding to positions of SEQ ID NO:2 may be readily determined.

Identification of the corresponding amino acid residue in another protease can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

In describing the variants of the present invention, the nomenclature described below is adapted for ease of reference. The accepted IUPAC single letter or three letter amino acid abbreviation is employed.

Substitutions: For an amino acid substitution, the following nomenclature is used: Original amino acid, position, substituted amino acid. Accordingly, the substitution of threonine at position 226 with alanine is designated as “Thr226Ala” or “T226A”. Multiple substitutions are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively. Alternatively, multiple substitutions may be separated by commas (“,”), e.g., “Gly205Arg,Ser411Phe” or “G205R,S411F.

Deletions: For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”. Alternatively, multiple deletions may be separated by commas (“,”), e.g., “Gly195*,Ser411*” or “G195*,S411*”.

Insertions: For an amino acid insertion, the following nomenclature is used: Original amino acid, position, original amino acid, inserted amino acid. Accordingly, the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lowercase letters to the position number of the amino acid residue preceding the inserted amino acid residue(s). In the above example, the sequence would thus be:

| Parent: | Variant: |
|---|---|
| 195 | 195 195a 195b |
| G | G - K - A |

Multiple alterations: Variants comprising multiple alterations are separated by addition marks (“+”), e.g., “Arg170Tyr+Gly195Glu” or “R170Y+G195E” representing a substitution of arginine and glycine at positions 170 and 195 with tyrosine and glutamic acid, respectively. Alternatively, multiple alterations may be separated by commas (“,”), e.g., “Arg170Tyr,Gly195Glu” or “R170Y,G195E”.

Different alterations: Where different alterations can be introduced at a position, the different alterations are separated by a comma, e.g., “Arg170Tyr,Glu” represents a substitution of arginine at position 170 with tyrosine or glutamic acid. Thus, “Tyr167Gly,Ala+Arg170Gly,Ala” designates the following variants:

### “Tyr167Gly+Arg170Gly”, “Tyr167Gly+Arg170Ala”, “Tyr167Ala+Arg170Gly”, and “Tyr167Ala+Arg170Ala”.

### SEQUENCE OVERVIEW

SEQ ID NO:1 is the amino acid sequence of the Savinase® protease.

SEQ ID NO:2 is the amino acid sequence of the BPN′ protease.

SEQ ID NO:3 is the amino acid sequence of a variant of SEQ ID NO:1.

SEQ ID NO:4 is the amino acid sequence of a variant of SEQ ID NO:1.

SEQ ID NO:5 is the amino acid sequence of a variant of SEQ ID NO:1.

SEQ ID NO:6 is the amino acid sequence of a variant of SEQ ID NO:1.

### DETAILED DESCRIPTION OF THE INVENTION

The present invention provides new protease variants with improved solubility. The protease variants of the invention comprise a positively charged or polar amino acid at a position corresponding to position 215 of SEQ ID NO:1 (i.e., position A215 of SEQ ID NO:1). The introduction of a positively charged or polar amino acid at this position results in improved solubility, in particular decreased protease crystal formation and increased protease crystal solubility, as described in the Examples below.

### Protease Variants

The present invention relates to a protease variant of a parent protease, wherein the variant has a sequence identity of at least at least 80%, at least 85%, at least 90%, at least 91%, at least 92%, at least 93%, at least 94%, at least 95%, at least 96%, at least 97%, at least 98%, or at least 99%, but less than 100%, to SEQ ID NO:1, wherein the variant comprises a first substitution selected from the group consisting of X215K, X215R, X215Q, X125N, X215S, and X215T, wherein the variant comprises at least three, e.g., at least four, at least five, at least six, at least seven, at least eight, at least nine, at least ten, or more, further alterations, preferably substitutions, selected from the group consisting of X3T (e.g., S3T), X4I (e.g., V4I), X9E (e.g., S9E), I35ID, X43R (e.g., N43R), X76D (e.g., N76D), X99D (e.g., S99D, X99F (e.g., S99F), X101E (e.g., S101E), X101L (e.g., S101L), X103A (e.g., S103A), X103T (e.g., S103T), X104I (e.g., V104I), X120D (e.g., H120D), X160S (e.g., G160S), X195E (e.g., G195E), X205I (e.g., V205I), X206L (e.g., Q206L), X209W (e.g., Y209W), X235L (e.g., K235L), X259D (e.g., S259D), X261W (e.g., N261W), and X262E (e.g., L262E), wherein the variant has protease activity, and wherein position numbers are based on the numbering of SEQ ID NO:2.

In an embodiment, the first substitution is selected from the group consisting of X215K, X215Q, X125N, X215S, and X215T; preferably the first substitution is selected from the group consisting X215K, X215Q, X125N, and X215T.

In an embodiment, the first substitution is selected from the group consisting of A215K, A215R, A215Q, A215N, A215S, and A215T; preferably, the first substitution is selected from the group consisting of A215K, A215Q, A215N, A215S, and A215T; most preferably, the first substitution is selected from the group consisting of A215K, A215Q, A215N, and A215T.

In an embodiment, the variant comprises at least three, e.g., at least four, at least five, at least six, at least seven, at least eight, at least nine, at least ten, or more, further alterations, preferably, substitutions selected from the group consisting of X3T (e.g., S3T), X4I (e.g., V4I), X9E (e.g., S9E), I35ID, X43R (e.g., N43R), X76D (e.g., N76D), X99D (e.g., S99D, X99F (e.g., S99F), X101E (e.g., S101E), X101L (e.g., S101L), X103A (e.g., S103A), X103T (e.g., S103T), X104I (e.g., V104I), X120D (e.g., H120D), X160S (e.g., G160S), X195E (e.g., G195E), X205I (e.g., V205I), X206L (e.g., Q206L), X209W (e.g., Y209W), X235L (e.g., K235L), X259D (e.g., S259D), X261W (e.g., N261W), and X262E (e.g., L262E). In a preferred embodiment, the variant comprises at least three, e.g., at least four, at least five, at least six, at least seven, at least eight, at least nine, at least ten, or more, further alterations, preferably substitutions, selected from the group consisting of S3T, V4I, S9E, I35ID, N43R, N76D, S99D, S99F, S101E, S101L, S103A, S103T, V104I, H120D, G160S, G195E, V205I, Q206L, Y209W, K235L, S259D, N261W, and L262E.

In a preferred embodiment, the protease variants comprise at least three, e.g., at least four, or five, further alterations, preferably substitutions, selected from a group of substitutions selected from the groups consisting of:
a) S3T, V4I, S99D, S101E, S103A, G160S, and V205I;
b) I35ID, N76D, H120D, G195E, K235L;
c) S9E, N43R, N76D, S99F, S101L, S103T, V104I, V205I, Q206L, Y209W, S259D, N261W, and L262E; and
d) S9E, N43R, N76D, V205I, Q206L, Y209W, S259D, N261W, and L262E.

In a preferred embodiment, the protease variants comprise at least three, e.g., at least four, at least five, at least six, or seven, further substitutions selected from the group consisting of S3T, V4I, S99D, S101E, S103A, G160S, and V205I.

In a preferred embodiment, the protease variants comprise at least three, e.g., at least four, or five, further alterations, preferably substitutions, selected from the group consisting of I35ID, N76D, H120D, G195E, and K235L.

In a preferred embodiment, the protease variants comprise at least three, e.g., at least four, at least five, at least six, at least seven, at least eight, at least nine, at least ten, at least eleven, at least twelve, or thirteen, further substitutions selected from the group consisting of S9E, N43R, N76D, S99F, S101L, S103T, V104I, V205I, Q206L, Y209W, S259D, N261W, and L262E.

In a preferred embodiment, the protease variants comprise at least three, e.g., at least four, at least five, at least six, at least seven, at least eight, or nine, further substitutions selected from the group consisting of S9E, N43R, N76D, V205I, Q206L, Y209W, S259D, N261W, and L262E.

In a preferred embodiment, the protease variant comprises, consists essentially of, or consists of SEQ ID NO:1 with a substitution selected from the group consisting of X215K, X215R, X215Q, X125N, X215S, and X215T; preferably a substitution selected from the group consisting of A215K, A215R, A215Q, A215N, A215S, and A215T; most preferably a substitution selected from the group consisting of A215K, A215Q, A215N, A215S, and A215T.

In a preferred embodiment, the protease variant comprises, consists essentially of, or consists of SEQ ID NO:3 with a substitution selected from the group consisting of X215K, X215R, X215Q, X125N, X215S, and X215T; preferably a substitution selected from the group consisting of A215K, A215R, A215Q, A215N, A215S, and A215T; most preferably a substitution selected from the group consisting of A215K, A215Q, A215N, A215S, and A215T.

In a preferred embodiment, the protease variant comprises, consists essentially of, or consists of SEQ ID NO:4 with a substitution selected from the group consisting of X215K, X215R, X215Q, X125N, X215S, and X215T; preferably a substitution selected from the group consisting of A215K, A215R, A215Q, A215N, A215S, and A215T; most preferably a substitution selected from the group consisting of A215K, A215Q, A215N, A215S, and A215T.

In a preferred embodiment, the protease variant comprises, consists essentially of, or consists of SEQ ID NO:5 with a substitution selected from the group consisting of X215K, X215R, X215Q, X125N, X215S, and X215T; preferably a substitution selected from the group consisting of A215K, A215R, A215Q, A215N, A215S, and A215T; most preferably a substitution selected from the group consisting of A215K, A215Q, A215N, A215S, and A215T.

In a preferred embodiment, the protease variant comprises, consists essentially of, or consists of SEQ ID NO:6 with a substitution selected from the group consisting of X215K, X215R, X215Q, X125N, X215S, and X215T; preferably a substitution selected from the group consisting of A215K, A215R, A215Q, A215N, A215S, and A215T; most preferably a substitution selected from the group consisting of A215K, A215Q, A215N, A215S, and A215T.

In addition to the substitutions described above, the variants may comprise further substitutions at one or more other positions.

The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a polyhistidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

The variants of the invention have improved solubility. In particular, the variants of the invention exhibit decreased protease crystal formation, e.g., during fermentation of host cells expressing the variants, as well as increased solubility of such protease crystals, as described in Example 1 below. Improved solubility may be determined in using various methods known to the skilled artisan. Preferably, improved solubility is determined as decreased protease crystal formation or increased protease crystal solubility according to Example 1 below.

In one embodiment, the protease variant has improved solubility compared to an otherwise identical protease without a substitution selected from the group consisting of X215K, X215R, X215Q, X125N, X215S, and X215T. In a preferred embodiment, the protease variant has improved solubility of at least 5%, e.g., at least 10%, at least 20%, at least 30%, at least 40%, at least 50%, at least 60%, at least 70%, at least 80%, at least 90%, at least 100%, at least 125%, at least 150%, at least 175%, at least 200%, at least 250%, at least 300%, at least 400%, at least 500%, or more, compared to an identical protease without the substitution selected from the group consisting of X215K, X215R, X215Q, X125N, X215S, and X215T and without the at least three further alterations, preferably substitutions, selected from the group consisting of X3T (e.g., S3T), X4I (e.g., V4I), X9E (e.g., S9E), I35ID, X43R (e.g., N43R), X76D (e.g., N76D), X99D (e.g., S99D, X99F (e.g., S99F), X101E (e.g., S101E), X101L (e.g., S101L), X103A (e.g., S103A), X103T (e.g., S103T), X104I (e.g., V104I), X120D (e.g., H120D), X160S (e.g., G160S), X195E (e.g., G195E), X205I (e.g., V205I), X206L (e.g., Q206L), X209W (e.g., Y209W), X235L (e.g., K235L), X259D (e.g., S259D), X261W (e.g., N261W), and X262E (e.g., L262E).

In a preferred embodiment, the protease variant has improved solubility at 10-30° C., preferably at 15-25° C., more preferably at about 20° C., most preferably at 20° C.

In a preferred embodiment, the protease variant has improved solubility at pH 3-9, preferably at pH 4-8, more preferably at pH 4-6, even more preferably at pH 4-5, most preferably at pH 4.5.

In a preferred embodiment, the protease variant has improved solubility at 15-25° C. and pH 4-6. Preferably, the protease variant has improved solubility at 20° C. and pH 4-5.

In one embodiment, the protease variant has improved solubility of at least 5%, e.g., at least 10%, at least 20%, at least 30%, at least 40%, at least 50%, at least 60%, at least 70%, at least 80%, at least 90%, at least 100%, at least 125%, at least 150%, at least 175%, at least 200%, at least 250%, at least 300%, at least 400%, at least 500%, or more, compared to SEQ ID NO:1.

In one embodiment, the protease variant has improved solubility of at least 5%, e.g., at least 10%, at least 20%, at least 30%, at least 40%, at least 50%, at least 60%, at least 70%, at least 80%, at least 90%, at least 100%, at least 125%, at least 150%, at least 175%, at least 200%, at least 250%, at least 300%, at least 400%, at least 500%, or more, compared to SEQ ID NO:3.

In one embodiment, the protease variant has improved solubility of at least 5%, e.g., at least 10%, at least 20%, at least 30%, at least 40%, at least 50%, at least 60%, at least 70%, at least 80%, at least 90%, at least 100%, at least 125%, at least 150%, at least 175%, at least 200%, at least 250%, at least 300%, at least 400%, at least 500%, or more, compared to SEQ ID NO:4.

In one embodiment, the protease variant has improved solubility of at least 5%, e.g., at least 10%, at least 20%, at least 30%, at least 40%, at least 50%, at least 60%, at least 70%, at least 80%, at least 90%, at least 100%, at least 125%, at least 150%, at least 175%, at least 200%, at least 250%, at least 300%, at least 400%, at least 500%, or more, compared to SEQ ID NO:5.

In one embodiment, the protease variant has improved solubility of at least 5%, e.g., at least 10%, at least 20%, at least 30%, at least 40%, at least 50%, at least 60%, at least 70%, at least 80%, at least 90%, at least 100%, at least 125%, at least 150%, at least 175%, at least 200%, at least 250%, at least 300%, at least 400%, at least 500%, or more, compared to SEQ ID NO:6.

In addition to improved solubility, the variants of the invention may have one or more improved properties compared to the parent. The one or more improved properties may be selected from the group consisting of catalytic efficiency, catalytic rate, chemical stability, oxidation stability, pH activity, pH stability, proteolytic stability, specific activity, stability under storage conditions, substrate binding, substrate cleavage, substrate specificity, substrate stability, surface properties, thermal activity, and thermostability.

The variants of the invention have protease activity, preferably on par or improved protease activity. In one embodiment, the protease variant has improved solubility compared to an otherwise identical protease without a substitution selected from the group consisting of X215K, X215R, X215Q, X125N, X215S, and X215T. In a preferred embodiment, the protease variant has on par or improved protease activity, e.g., at least 100%, at least 101%, at least 102%, at least 103%, at least 104%, at least 105%, at least 110%, at least 120%, at least 130%, at least 140%, at least 150%, at least 175%, at least 200%, at least 250%, at least 300%, at least 400%, at least 500%, compared to an identical protease without the first substitution selected from the group consisting of X215K, X215R, X215Q, X125N, X215S, and X215T and without the at least three further alterations, preferably substitutions, selected from the group consisting of X3T (e.g., S3T), X4I (e.g., V4I), X9E (e.g., S9E), I35ID, X43R (e.g., N43R), X76D (e.g., N76D), X99D (e.g., S99D, X99F (e.g., S99F), X101E (e.g., S101E), X101L (e.g., S101L), X103A (e.g., S103A), X103T (e.g., S103T), X104I (e.g., V104I), X120D (e.g., H120D), X160S (e.g., G160S), X195E (e.g., G195E), X205I (e.g., V205I), X206L (e.g., Q206L), X209W (e.g., Y209W), X235L (e.g., K235L), X259D (e.g., S259D), X261W (e.g., N261W), and X262E (e.g., L262E).

In one embodiment, the variants have on par or improved protease activity, e.g., at least 100%, at least 101%, at least 102%, at least 103%, at least 104%, at least 105%, at least 110%, at least 120%, at least 130%, at least 140%, at least 150%, at least 175%, at least 200%, at least 250%, at least 300%, at least 400%, at least 500%, or more, compared to the protease activity of SEQ ID NO:1.

In one embodiment, the variants have on par or improved protease activity, e.g., at least 100%, at least 101%, at least 102%, at least 103%, at least 104%, at least 105%, at least 110%, at least 120%, at least 130%, at least 140%, at least 150%, at least 175%, at least 200%, at least 250%, at least 300%, at least 400%, at least 500%, or more, compared to the protease activity of SEQ ID NO:3.

In one embodiment, the variants have on par or improved protease activity, e.g., at least 100%, at least 101%, at least 102%, at least 103%, at least 104%, at least 105%, at least 110%, at least 120%, at least 130%, at least 140%, at least 150%, at least 175%, at least 200%, at least 250%, at least 300%, at least 400%, at least 500%, or more, compared to the protease activity of SEQ ID NO:4.

In one embodiment, the variants have on par or improved protease activity, e.g., at least 100%, at least 101%, at least 102%, at least 103%, at least 104%, at least 105%, at least 110%, at least 120%, at least 130%, at least 140%, at least 150%, at least 175%, at least 200%, at least 250%, at least 300%, at least 400%, at least 500%, or more, compared to the protease activity of SEQ ID NO:5.

In one embodiment, the variants have on par or improved protease activity, e.g., at least 100%, at least 101%, at least 102%, at least 103%, at least 104%, at least 105%, at least 110%, at least 120%, at least 130%, at least 140%, at least 150%, at least 175%, at least 200%, at least 250%, at least 300%, at least 400%, at least 500%, or more, compared to the protease activity of SEQ ID NO:6.

In one aspect, the present invention relates to a polypeptide, preferably an isolated or purified polypeptide, having a sequence identity of at least 80%, e.g., at least 85%, at least 90%, at least 91%, at least 92%, at least 93%, at least 94%, at least 95%, at least 96%, at least 97%, at least 98%, at least 99%, but less than 100%, to SEQ ID NO:1, wherein the variant comprises a substitution selected from the group consisting of X215K, X215R, X215Q, X125N, X215S, and X215T, wherein the variant has protease activity, and wherein position numbers are based on the numbering of SEQ ID NO:2. In a preferred embodiment, the variant comprises a substitution selected from the group consisting of A215K, A215R, A215Q, A125N, A215S, and A215T. In a preferred embodiment, the variant comprises a substitution selected from the group consisting of A215K, A215Q, A215N, A215S, and A215T. In a preferred embodiment, the variant comprises a substitution selected from the group consisting of A215K, A215Q, A215N, and A215T. In one embodiment, the variant has on par or improved protease activity, e.g., at least 100%, at least 101%, at least 102%, at least 103%, at least 104%, at least 105%, at least 110%, at least 120%, at least 130%, at least 140%, at least 150%, at least 175%, at least 200%, at least 250%, at least 300%, at least 400%, at least 500%, or more, compared to SEQ ID NO:1. In one embodiment, the variant has improved solubility of at least 5%, e.g., at least 10%, at least 20%, at least 30%, at least 40%, at least 50%, at least 60%, at least 70%, at least 80%, at least 90%, at least 100%, at least 125%, at least 150%, at least 175%, at least 200%, at least 250%, at least 300%, at least 400%, at least 500%, or more, compared to SEQ ID NO:1. In a preferred embodiment, the variant has improved solubility at 10-30° C., preferably at 15-25° C., more preferably at about 20° C., most preferably at 20° C. In a preferred embodiment, the variant has improved solubility at pH 3-9, preferably at pH 4-8, more preferably at pH 4-6, even more preferably at pH 4-5, most preferably at pH 4.5. In a preferred embodiment, the variant comprises, consists essentially of, or consists of SEQ ID NO:1 with the substitution A215K. In a preferred embodiment, the variant comprises, consists essentially of, or consists of SEQ ID NO:1 with the substitution A215R. In a preferred embodiment, the variant comprises, consists essentially of, or consists of SEQ ID NO:1 with the substitution A215Q. In a preferred embodiment, the variant comprises, consists essentially of, or consists of SEQ ID NO:1 with the substitution A215N. In a preferred embodiment, the variant comprises, consists essentially of, or consists of SEQ ID NO:1 with the substitution A215S. In a preferred embodiment, the variant comprises, consists essentially of, or consists of SEQ ID NO:1 with the substitution A215T.

In one aspect, the present invention relates to a polypeptide, preferably an isolated or purified polypeptide, having a sequence identity of at least 80%, e.g., at least 85%, at least 90%, at least 91%, at least 92%, at least 93%, at least 94%, at least 95%, at least 96%, at least 97%, at least 98%, at least 99%, but less than 100%, to SEQ ID NO:3, wherein the variant comprises a first substitution selected from the group consisting of X215K, X215R, X215Q, X125N, X215S, and X215T, wherein the variants comprise at least three, e.g., at least four, at least five, at least six, or seven, further substitutions selected from the group consisting of S3T, V4I, S99D, S101E, S103A, G160S, and V205I, wherein the variant has protease activity, and wherein position numbers are based on the numbering of SEQ ID NO:2. In a preferred embodiment, the variant comprises a first substitution selected from the group consisting of A215K, A215R, A215Q, A125N, A215S, and A215T. In a preferred embodiment, the variant comprises a first substitution selected from the group consisting of A215K, A215Q, A215N, A215S, and A215T. In a preferred embodiment, the variant comprises a first substitution selected from the group consisting of A215K, A215Q, A215N, and A215T. In one embodiment, the variant has on par or improved protease activity, e.g., at least 100%, at least 101%, at least 102%, at least 103%, at least 104%, at least 105%, at least 110%, at least 120%, at least 130%, at least 140%, at least 150%, at least 175%, at least 200%, at least 250%, at least 300%, at least 400%, at least 500%, or more, compared to SEQ ID NO:3. In one embodiment, the variant has improved solubility of at least 5%, e.g., at least 10%, at least 20%, at least 30%, at least 40%, at least 50%, at least 60%, at least 70%, at least 80%, at least 90%, at least 100%, at least 125%, at least 150%, at least 175%, at least 200%, at least 250%, at least 300%, at least 400%, at least 500%, or more, compared to SEQ ID NO:3. In a preferred embodiment, the variant has improved solubility at 10-30° C., preferably at 15-25° C., more preferably at about 20° C., most preferably at 20° C. In a preferred embodiment, the variant has improved solubility at pH 3-9, preferably at pH 4-8, more preferably at pH 4-6, even more preferably at pH 4-5, most preferably at pH 4.5. In a preferred embodiment, the variant comprises, consists essentially of, or consists of SEQ ID NO:3 with the substitution A215K. In a preferred embodiment, the variant comprises, consists essentially of, or consists of SEQ ID NO:3 with the substitution A215R. In a preferred embodiment, the variant comprises, consists essentially of, or consists of SEQ ID NO:3 with the substitution A215Q. In a preferred embodiment, the variant comprises, consists essentially of, or consists of SEQ ID NO:3 with the substitution A215N. In a preferred embodiment, the variant comprises, consists essentially of, or consists of SEQ ID NO:3 with the substitution A215S. In a preferred embodiment, the variant comprises, consists essentially of, or consists of SEQ ID NO:3 with the substitution A215T.

In one aspect, the present invention relates to a polypeptide, preferably an isolated or purified polypeptide, having a sequence identity of at least 80%, e.g., at least 85%, at least 90%, at least 91%, at least 92%, at least 93%, at least 94%, at least 95%, at least 96%, at least 97%, at least 98%, at least 99%, but less than 100%, to SEQ ID NO:4, wherein the variant comprises a first substitution selected from the group consisting of X215K, X215R, X215Q, X125N, X215S, and X215T, wherein the variants comprise at least three, e.g., at least four, or five, further substitutions selected from the group consisting of I35ID, N76D, H120D, G195E, and K235L, wherein the variant has protease activity, and wherein position numbers are based on the numbering of SEQ ID NO:2. In a preferred embodiment, the variant comprises a first substitution selected from the group consisting of A215K, A215R, A215Q, A125N, A215S, and A215T. In a preferred embodiment, the variant comprises a first substitution selected from the group consisting of A215K, A215Q, A215N, A215S, and A215T. In a preferred embodiment, the variant comprises a first substitution selected from the group consisting of A215K, A215Q, A215N, and A215T. In one embodiment, the variant has on par or improved protease activity, e.g., at least 100%, at least 101%, at least 102%, at least 103%, at least 104%, at least 105%, at least 110%, at least 120%, at least 130%, at least 140%, at least 150%, at least 175%, at least 200%, at least 250%, at least 300%, at least 400%, at least 500%, or more, compared to SEQ ID NO:4. In one embodiment, the variant has improved solubility of at least 5%, e.g., at least 10%, at least 20%, at least 30%, at least 40%, at least 50%, at least 60%, at least 70%, at least 80%, at least 90%, at least 100%, at least 125%, at least 150%, at least 175%, at least 200%, at least 250%, at least 300%, at least 400%, at least 500%, or more, compared to SEQ ID NO:4. In a preferred embodiment, the variant has improved solubility at 10-30° C., preferably at 15-25° C., more preferably at about 20° C., most preferably at 20° C. In a preferred embodiment, the variant has improved solubility at pH 3-9, preferably at pH 4-8, more preferably at pH 4-6, even more preferably at pH 4-5, most preferably at pH 4.5. In a preferred embodiment, the variant comprises, consists essentially of, or consists of SEQ ID NO:4 with the substitution A215K. In a preferred embodiment, the variant comprises, consists essentially of, or consists of SEQ ID NO:4 with the substitution A215R. In a preferred embodiment, the variant comprises, consists essentially of, or consists of SEQ ID NO:4 with the substitution A215Q. In a preferred embodiment, the variant comprises, consists essentially of, or consists of SEQ ID NO:4 with the substitution A215N. In a preferred embodiment, the variant comprises, consists essentially of, or consists of SEQ ID NO:4 with the substitution A215S. In a preferred embodiment, the variant comprises, consists essentially of, or consists of SEQ ID NO:4 with the substitution A215T.

In one aspect, the present invention relates to a polypeptide, preferably an isolated or purified polypeptide, having a sequence identity of at least 80%, e.g., at least 85%, at least 90%, at least 91%, at least 92%, at least 93%, at least 94%, at least 95%, at least 96%, at least 97%, at least 98%, at least 99%, but less than 100%, to SEQ ID NO:5, wherein the variant comprises a first substitution selected from the group consisting of X215K, X215R, X215Q, X125N, X215S, and X215T, wherein the variants comprise at least three, e.g., at least four, at least five, at least six, at least seven, at least eight, at least nine, at least ten, at least eleven, at least twelve, or thirteen, further substitutions selected from the group consisting of S9E, N43R, N76D, S99F, S101L, S103T, V104I, V205I, Q206L, Y209W, S259D, N261W, and L262E, wherein the variant has protease activity, and wherein position numbers are based on the numbering of SEQ ID NO:2. In a preferred embodiment, the variant comprises a first substitution selected from the group consisting of A215K, A215R, A215Q, A125N, A215S, and A215T. In a preferred embodiment, the variant comprises first a substitution selected from the group consisting of A215K, A215Q, A215N, A215S, and A215T. In a preferred embodiment, the variant comprises first a substitution selected from the group consisting of A215K, A215Q, A215N, and A215T. In one embodiment, the variant has on par or improved protease activity, e.g., at least 100%, at least 101%, at least 102%, at least 103%, at least 104%, at least 105%, at least 110%, at least 120%, at least 130%, at least 140%, at least 150%, at least 175%, at least 200%, at least 250%, at least 300%, at least 400%, at least 500%, or more, compared to SEQ ID NO:5. In one embodiment, the variant has improved solubility of at least 5%, e.g., at least 10%, at least 20%, at least 30%, at least 40%, at least 50%, at least 60%, at least 70%, at least 80%, at least 90%, at least 100%, at least 125%, at least 150%, at least 175%, at least 200%, at least 250%, at least 300%, at least 400%, at least 500%, or more, compared to SEQ ID NO:5. In a preferred embodiment, the variant has improved solubility at 10-30° C., preferably at 15-25° C., more preferably at about 20° C., most preferably at 20° C. In a preferred embodiment, the variant has improved solubility at pH 3-9, preferably at pH 4-8, more preferably at pH 4-6, even more preferably at pH 4-5, most preferably at pH 4.5. In a preferred embodiment, the variant comprises, consists essentially of, or consists of SEQ ID NO:5 with the substitution A215K. In a preferred embodiment, the variant comprises, consists essentially of, or consists of SEQ ID NO:5 with the substitution A215R. In a preferred embodiment, the variant comprises, consists essentially of, or consists of SEQ ID NO:5 with the substitution A215Q. In a preferred embodiment, the variant comprises, consists essentially of, or consists of SEQ ID NO:5 with the substitution A215N. In a preferred embodiment, the variant comprises, consists essentially of, or consists of SEQ ID NO:5 with the substitution A215S. In a preferred embodiment, the variant comprises, consists essentially of, or consists of SEQ ID NO:5 with the substitution A215T.

In one aspect, the present invention relates to a polypeptide, preferably an isolated or purified polypeptide, having a sequence identity of at least 80%, e.g., at least 85%, at least 90%, at least 91%, at least 92%, at least 93%, at least 94%, at least 95%, at least 96%, at least 97%, at least 98%, at least 99%, but less than 100%, to SEQ ID NO:6, wherein the variant comprises a first substitution selected from the group consisting of X215K, X215R, X215Q, X125N, X215S, and X215T, wherein the variants comprise at least three, e.g., at least four, at least five, at least six, at least seven, at least eight, or nine, further substitutions selected from the group consisting of S9E, N43R, N76D, V205I, Q206L, Y209W, S259D, N261W, and L262E, wherein the variant has protease activity, and wherein position numbers are based on the numbering of SEQ ID NO:2. In a preferred embodiment, the variant comprises a first substitution selected from the group consisting of A215K, A215R, A215Q, A125N, A215S, and A215T. In a preferred embodiment, the variant comprises first a substitution selected from the group consisting of A215K, A215Q, A215N, A215S, and A215T. In a preferred embodiment, the variant comprises first a substitution selected from the group consisting of A215K, A215Q, A215N, and A215T. In one embodiment, the variant has on par or improved protease activity, e.g., at least 100%, at least 101%, at least 102%, at least 103%, at least 104%, at least 105%, at least 110%, at least 120%, at least 130%, at least 140%, at least 150%, at least 175%, at least 200%, at least 250%, at least 300%, at least 400%, at least 500%, or more, compared to SEQ ID NO:6. In one embodiment, the variant has improved solubility of at least 5%, e.g., at least 10%, at least 20%, at least 30%, at least 40%, at least 50%, at least 60%, at least 70%, at least 80%, at least 90%, at least 100%, at least 125%, at least 150%, at least 175%, at least 200%, at least 250%, at least 300%, at least 400%, at least 500%, or more, compared to SEQ ID NO:6. In a preferred embodiment, the variant has improved solubility at 10-30° C., preferably at 15-25° C., more preferably at about 20° C., most preferably at 20° C. In a preferred embodiment, the variant has improved solubility at pH 3-9, preferably at pH 4-8, more preferably at pH 4-6, even more preferably at pH 4-5, most preferably at pH 4.5. In a preferred embodiment, the variant comprises, consists essentially of, or consists of SEQ ID NO:6 with the substitution A215K. In a preferred embodiment, the variant comprises, consists essentially of, or consists of SEQ ID NO:6 with the substitution A215R. In a preferred embodiment, the variant comprises, consists essentially of, or consists of SEQ ID NO:6 with the substitution A215Q. In a preferred embodiment, the variant comprises, consists essentially of, or consists of SEQ ID NO:6 with the substitution A215N. In a preferred embodiment, the variant comprises, consists essentially of, or consists of SEQ ID NO:6 with the substitution A215S. In a preferred embodiment, the variant comprises, consists essentially of, or consists of SEQ ID NO:6 with the substitution A215T.

### Parent Proteases

Protease variants of the invention may be based on any parent protease. The parent may be a naturally occurring (wild-type) polypeptide or a variant or fragment thereof.

In one aspect, the parent protease has a sequence identity to the polypeptide of SEQ ID NO:1 of at least 80%, e.g., at least 85%, at least 90%, at least 91%, at least 92%, at least 93%, at least 94%, at least 95%, at least 96%, at least 97%, at least 98%, at least 99%, or 100%, and has protease activity. In an embodiment, the amino acid sequence of the parent differs by up to amino acids, e.g., 1, 2, 3, 4, 5, 6, 7, 8, 9, 10, 11, 12, 13, 14, 15, 16, 17, 18, 19, or 20, from the polypeptide of SEQ ID NO:1. In an embodiment, the parent comprises, consists essentially of, or consists of the amino acid sequence of SEQ ID NO:1.

In one aspect, the parent protease has a sequence identity to the polypeptide of SEQ ID NO:3 of at least 80%, e.g., at least 85%, at least 90%, at least 91%, at least 92%, at least 93%, at least 94%, at least 95%, at least 96%, at least 97%, at least 98%, at least 99%, or 100%, and has protease activity. In an embodiment, the amino acid sequence of the parent differs by up to amino acids, e.g., 1, 2, 3, 4, 5, 6, 7, 8, 9, 10, 11, 12, 13, 14, 15, 16, 17, 18, 19, or 20, from the polypeptide of SEQ ID NO:3. In an embodiment, the parent comprises, consists essentially of, or consists of the amino acid sequence of SEQ ID NO:3.

In one aspect, the parent protease has a sequence identity to the polypeptide of SEQ ID NO:4 of at least 80%, e.g., at least 85%, at least 90%, at least 91%, at least 92%, at least 93%, at least 94%, at least 95%, at least 96%, at least 97%, at least 98%, at least 99%, or 100%, and has protease activity. In an embodiment, the amino acid sequence of the parent differs by up to amino acids, e.g., 1, 2, 3, 4, 5, 6, 7, 8, 9, 10, 11, 12, 13, 14, 15, 16, 17, 18, 19, or 20, from the polypeptide of SEQ ID NO:4. In an embodiment, the parent comprises, consists essentially of, or consists of the amino acid sequence of SEQ ID NO:4.

In one aspect, the parent protease has a sequence identity to the polypeptide of SEQ ID NO:5 of at least 80%, e.g., at least 85%, at least 90%, at least 91%, at least 92%, at least 93%, at least 94%, at least 95%, at least 96%, at least 97%, at least 98%, at least 99%, or 100%, and has protease activity. In an embodiment, the amino acid sequence of the parent differs by up to amino acids, e.g., 1, 2, 3, 4, 5, 6, 7, 8, 9, 10, 11, 12, 13, 14, 15, 16, 17, 18, 19, or 20, from the polypeptide of SEQ ID NO:5. In an embodiment, the parent comprises, consists essentially of, or consists of the amino acid sequence of SEQ ID NO:5.

In one aspect, the parent protease has a sequence identity to the polypeptide of SEQ ID NO:6 of at least 80%, e.g., at least 85%, at least 90%, at least 91%, at least 92%, at least 93%, at least 94%, at least 95%, at least 96%, at least 97%, at least 98%, at least 99%, or 100%, and has protease activity. In an embodiment, the amino acid sequence of the parent differs by up to amino acids, e.g., 1, 2, 3, 4, 5, 6, 7, 8, 9, 10, 11, 12, 13, 14, 15, 16, 17, 18, 19, or 20, from the polypeptide of SEQ ID NO:6. In an embodiment, the parent comprises, consists essentially of, or consists of the amino acid sequence of SEQ ID NO:6.

The parent polypeptide may be a hybrid polypeptide in which a region of one polypeptide is fused at the N-terminus or the C-terminus of a region of another polypeptide.

The parent may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

The parent may be obtained from microorganisms of any genus. For purposes of the present invention, the term “obtained from” as used herein in connection with a given source shall mean that the parent encoded by a polynucleotide is produced by the source or by a strain in which the polynucleotide from the source has been inserted. In one aspect, the parent is secreted extracellularly.

The parent may be a bacterial protease. For example, the parent may be a Gram-positive bacterial polypeptide such as a Bacillus, Clostridium, Enterococcus, Geobacillus, Lactobacillus, Lactococcus, Oceanobacillus, Staphylococcus, Streptococcus, or Streptomyces protease, or a Gram-negative bacterial polypeptide such as a Campylobacter, E. coli, Flavobacterium, Fusobacterium, Helicobacter, Ilyobacter, Neisseria, Pseudomonas, Salmonella, or Ureaplasma protease.

In one aspect, the parent is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis protease.

In another aspect, the parent is a Streptococcus equisimilis, Streptococcus pyogenes, Streptococcus uberis, or Streptococcus equi subsp. Zooepidemicus protease.

In another aspect, the parent is a Streptomyces achromogenes, Streptomyces avermitilis, Streptomyces coelicolor, Streptomyces griseus, or Streptomyces lividans protease.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The parent may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding a parent may then be obtained by similarly screening a genomic DNA or cDNA library of another microorganism or mixed DNA sample. Once a polynucleotide encoding a parent has been detected with the probe(s), the polynucleotide can be isolated or cloned by utilizing techniques that are known to those of ordinary skill in the art (see, e.g., Sambrook et al., 1989).

### Preparation of Protease Variants

The present invention also relates to methods for obtaining a protease variant, the method comprising:

(a) introducing into a parent protease a first substitution selected from the group consisting of X215K, X215R, X215Q, X125N, X215S, and X215T; and introducing at least three further alterations, preferably substitutions, selected from the group consisting of X3T (e.g., S3T), X4I (e.g., V4I), X9E (e.g., S9E), I35ID, X43R (e.g., N43R), X76D (e.g., N76D), X99D (e.g., S99D, X99F (e.g., S99F), X101E (e.g., S101E), X101L (e.g., S101L), X103A (e.g., S103A), X103T (e.g., S103T), X104I (e.g., V104I), X120D (e.g., H120D), X160S (e.g., G160S), X195E (e.g., G195E), X205I (e.g., V205I), X206L (e.g., Q206L), X209W (e.g., Y209W), X235L (e.g., K235L), X259D (e.g., S259D), X261W (e.g., N261W), and X262E (e.g., L262E); wherein the variant has protease activity; and

(b) recovering the variant.

In one embodiment, the first substitution is selected from the group consisting of X215K, X215Q, X125N, X215S, and X215T; preferably the first substitution is selected from the group consisting of X215K, X215Q, X125N, and X215T.

In one embodiment, first substitution is selected from the group consisting of A215K, A215R, A215Q, A215N, A215S, and A215T; preferably the first substitution is selected from the group consisting of A215K, A215Q, A215N, A215S, and A215T; most preferably the first substitution is selected from the group consisting of A215K, A215Q, A215N, and A215T.

In one embodiment, the at least three further alterations, preferably substitutions, are selected from the group consisting of S3T, V4I, S9E, I35ID, N43R, N76D, S99D, S99F, S101E, S101L, S103A, S103T, V104I, H120D, G160S, G195E, V205I, Q206L, Y209W, K235L, S259D, N261W, and L262E.

In one embodiment, the at least three further alterations, preferably substitutions, are selected from one of the groups consisting of:
a) S3T, V4I, S99D, S101E, S103A, G160S, and V205I;
b) I35ID, N76D, H120D, G195E, K235L;
c) S9E, N43R, N76D, S99F, S101L, S103T, V104I, V205I, Q206L, Y209W, S259D, N261W, and L262E; and
d) S9E, N43R, N76D, V205I, Q206L, Y209W, S259D, N261W, and L262E.

The variants can be prepared using any mutagenesis procedure known in the art, such as site-directed mutagenesis, synthetic gene construction, semi-synthetic gene construction, random mutagenesis, shuffling, etc.

Site-directed mutagenesis is a technique in which one or more mutations are introduced at one or more defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76: 4949-4955; and Barton et al., 1990, Nucleic Acids Res. 18: 7349-4966.

Site-directed mutagenesis can also be accomplished in vivo by methods known in the art. See, e.g., U.S. Patent Application Publication No. 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16.

Any site-directed mutagenesis procedure can be used in the present invention. There are many commercial kits available that can be used to prepare variants.

Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

### Polynucleotides

The present invention also relates to isolated polynucleotides encoding a variant of the present invention.

The techniques used to isolate or clone a polynucleotide are known in the art and include isolation from genomic DNA or cDNA, or a combination thereof. The cloning of the polynucleotides from genomic DNA can be achieved, e.g., by using the polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used.

### Nucleic Acid Constructs

The present invention also relates to nucleic acid constructs comprising a polynucleotide encoding a variant of the present invention operably linked to one or more control sequences that direct the expression of the coding sequence in a suitable host cell under conditions compatible with the control sequences.

The polynucleotide may be manipulated in a variety of ways to provide for expression of a variant. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide recognized by a host cell for expression of a polynucleotide encoding a variant of the present invention. The promoter contains transcriptional control sequences that mediate the expression of the variant. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the variant. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a non-translated region of an mRNA that is important for translation by the host cell. The leader is operably linked to the 5′-terminus of the polynucleotide encoding the variant. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a variant and directs the variant into the cell's secretory pathway. The 5′-end of the coding sequence of the polynucleotide may inherently contain a signal peptide coding sequence naturally linked in translation reading frame with the segment of the coding sequence that encodes the variant. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the variant. However, any signal peptide coding sequence that directs the expressed variant into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a variant. The resultant polypeptide is known as a proenzyme or propolypeptide (or a zymogen in some cases). A propolypeptide is generally inactive and can be converted to an active variant by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a variant and the signal peptide sequence is positioned next to the N-terminus of the propeptide sequence.

It may also be desirable to add regulatory sequences that regulate expression of the variant relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the variant would be operably linked to the regulatory sequence.

### Expression Vectors

The present invention also relates to recombinant expression vectors comprising a polynucleotide encoding a variant of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosylaminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is a hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the variant or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell.

Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMß1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a variant. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989).

### Host Cells

The present invention also relates to recombinant host cells, comprising a polynucleotide encoding a variant of the present invention operably linked to one or more control sequences that direct the production of a variant of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the variant and its source.

The host cell may be any cell useful in the recombinant production of a variant, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells. Preferably, the bacterial host cell is a Bacillus licheniformis cell.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell, including, but not limited to, Streptomyces achromogenes, Streptomyces avermitilis, Streptomyces coelicolor, Streptomyces griseus, and Streptomyces lividans cells.

The introduction of DNA into a Bacillus cell may be achieved by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be achieved by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be achieved by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be achieved by electroporation (see, e.g., Choi et al., 2006, J. Microbiol. Methods 64: 391-397), or conjugation (see, e.g., Pinedo and Smets, 2005, Appl. Environ. Microbiol. 71: 51-57). The introduction of DNA into a Streptococcus cell may be achieved by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a variant, comprising (a) cultivating a recombinant host cell of the present invention under conditions conducive for production of the variant; and optionally (b) recovering the variant.

The recombinant host cells are cultivated in a nutrient medium suitable for production of the variant using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the variant to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the variant is secreted into the nutrient medium, the variant can be recovered directly from the medium. If the variant is not secreted, it can be recovered from cell lysates.

The variants may be detected using methods known in the art that are specific for the variants. These detection methods include, but are not limited to, use of specific antibodies, formation of an enzyme product, or disappearance of an enzyme substrate. For example, an enzyme assay may be used to determine the activity of the variant.

The variant may be recovered using methods known in the art. For example, the variant may be recovered from the nutrient medium by conventional procedures including, but not limited to, collection, centrifugation, filtration, extraction, spray-drying, evaporation, or precipitation. In one aspect, the whole fermentation broth is recovered.

The variant may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure variants.

In an alternative aspect, the variant is not recovered, but rather a host cell of the present invention expressing the variant is used as a source of the variant.

### Fermentation Broth Formulations or Cell Compositions

The present invention also relates to a fermentation broth formulation or a cell composition comprising a variant of the present invention. The fermentation broth product further comprises additional ingredients used in the fermentation process, such as, for example, cells (including, the host cells containing the gene encoding the variant of the present invention which are used to produce the variant of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

### Detergent Compositions

The present invention also relates to a composition comprising a protease variant of the invention, e.g., a detergent or cleaning composition.

The invention also relates to a composition comprising a protease variant of the invention and further comprising one or more detergent components and/or one or more additional enzymes. In a preferred embodiment, the composition is a detergent composition comprising one or more detergent components, in particular one or more non-naturally occurring detergent components.

The present invention also relates to a composition comprising a protease variant of the present invention and further comprising one or more additional enzymes selected from the group consisting of amylases, catalases, cellulases (e.g., endoglucanases), cutinases, haloperoxygenases, lipases, mannanases, pectinases, pectin lyases, peroxidases, proteases, xanthanases, lichenases and xyloglucanases, or any mixture thereof.

A detergent composition may, e.g., be in the form of a bar, a homogeneous tablet, a tablet having two or more layers, a pouch having one or more compartments, a regular or compact powder, a granule, a paste, a gel, or a regular, compact or concentrated liquid. In a preferred embodiment, the detergent composition is in the form of a liquid or gel, in particular a liquid laundry detergent.

The invention also relates to use of a composition of the present in a cleaning process, such as laundry or hard surface cleaning such as dish wash.

The choice of additional components for a detergent composition is within the skill of the artisan and includes conventional ingredients, including the exemplary non-limiting components set forth below. The choice of components may include, for fabric care, the consideration of the type of fabric to be cleaned, the type and/or degree of soiling, the temperature at which cleaning is to take place, and the formulation of the detergent product.

In a particular embodiment, a detergent composition comprises a protease variant of the invention and one or more non-naturally occurring detergent components, such as surfactants, hydrotropes, builders, co-builders, chelators or chelating agents, bleaching system or bleach components, polymers, fabric hueing agents, fabric conditioners, foam boosters, suds suppressors, dispersants, dye transfer inhibitors, fluorescent whitening agents, perfume, optical brighteners, bactericides, fungicides, soil suspending agents, soil release polymers, anti-redeposition agents, enzyme inhibitors or stabilizers, enzyme activators, antioxidants, and solubilizers.

In one embodiment, the protease variant of the invention may be added to a detergent composition in an amount corresponding to 0.01-200 mg of enzyme protein per liter of wash liquor, preferably 0.05-50 mg of enzyme protein per liter of wash liquor, in particular 0.1-10 mg of enzyme protein per liter of wash liquor.

An automatic dish wash (ADW) composition may for example include 0.001%-30%, such as 0.01%-20%, such as 0.1-15%, such as 0.5-10% of enzyme protein by weight of the composition.

A granulated composition for laundry may for example include 0.001%-20%, such as 0.01%-10%, such as 0.05%-5% of enzyme protein by weight of the composition.

A liquid composition for laundry may for example include 0.0001%-10%, such as 0.001-7%, such as 0.1%-5% of enzyme protein by weight of the composition.

The enzymes such as the protease variant of the invention may be stabilized using conventional stabilizing agents, e.g., a polyol such as propylene glycol or glycerol, a sugar or sugar alcohol, lactic acid, boric acid, or a boric acid derivative, e.g., an aromatic borate ester, or a phenyl boronic acid derivative such as 4-formylphenyl boronic acid, and the composition may be formulated as described in, for example, WO 1992/19709 and WO 1992/19708 or the variants according to the invention may be stabilized using peptide aldehydes or ketones such as described in WO 2005/105826 and WO 2009/118375.
The protease variants of the invention may be formulated in liquid laundry compositions such as a liquid laundry compositions composition comprising:

a) at least 0.01 mg of active protease variant per litre detergent,

b) 2 wt % to 60 wt % of at least one surfactant

c) 5 wt % to 50 wt % of at least one builder

The detergent composition may be formulated into a granular detergent for laundry. Such detergent may comprise;

a) at least 0.01 mg of active protease variant per gram of composition

b) anionic surfactant, preferably 5 wt % to 50 wt %

c) nonionic surfactant, preferably 1 wt % to 8 wt %

d) builder, preferably 5 wt % to 40 wt %, such as carbonates, zeolites, phosphate builder, calcium sequestering builders or complexing agents.

Although components mentioned below are categorized by general header according to a particular functionality, this is not to be construed as a limitation, as a component may comprise additional functionalities as will be appreciated by the person skilled in the art.

### Surfactants

The detergent composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant(s) is chosen based on the desired cleaning application, and includes any conventional surfactant(s) known in the art. Any surfactant known in the art for use in detergents may be utilized. Surfactants lower the surface tension in the detergent, which allows the stain being cleaned to be lifted and dispersed and then washed away.

When included therein, the detergent will usually contain from about 1% to about 40% by weight, such as from about 5% to about 30%, including from about 5% to about 15%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or soap, and combinations thereof.

When included therein, the detergent will usually contain from about 0% to about 10% by weight of a cationic surfactant. Non-limiting examples of cationic surfactants include alklydimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, and combinations thereof.

When included therein, the detergent will usually contain from about 0.2% to about 40% by weight of a non-ionic surfactant, for example from about 0.5% to about 30%, in particular from about 1% to about 20%, from about 3% to about 10%, such as from about 3% to about 5%, or from about 8% to about 12%. Non-limiting examples of non-ionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxy alkyl fatty acid amides, or N-acyl N-alkyl derivatives of glucosamine (glucamides, GA, or fatty acid glucamide, FAGA), as well as products available under the trade names SPAN and TWEEN, and combinations thereof.

When included therein, the detergent will usually contain from about 0% to about 10% by weight of a semipolar surfactant. Non-limiting examples of semipolar surfactants include amine oxides (AO) such as alkyldimethylamineoxide, N-(coco alkyl)-N,N-dimethylamine oxide and N(tallow-alkyl)-N,N-bis(2-hydroxyethyl)amine oxide, fatty acid alkanolamides and ethoxylated fatty acid alkanolamides, and combinations thereof.

When included therein, the detergent will usually contain from about 0% to about 10% by weight of a zwitterionic surfactant. Non-limiting examples of zwitterionic surfactants include betaine, alkyldimethylbetaine, sulfobetaine, and combinations thereof.

### Builders and Co-Builders

The detergent composition may contain about 0-65% by weight, such as about 5% to about 45% of a detergent builder or co-builder, or a mixture thereof. In a dish wash detergent, the level of builder is typically 40-65%, particularly 50-65%. Builders and chelators soften, e.g., the wash water by removing the metal ions form the liquid. The builder and/or co-builder may particularly be a chelating agent that forms water-soluble complexes with Ca and Mg. Any builder and/or co-builder known in the art for use in laundry detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as iminodiethanol), triethanolamine (TEA, also known as 2,2′,2″-nitrilotriethanol), and carboxymethyl inulin (CMI), and combinations thereof.

The detergent composition may also contain 0-20% by weight, such as about 5% to about 10%, of a detergent co-builder, or a mixture thereof. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra-(methylenephosphonic acid) (EDTMPA), diethylenetriaminepentakis (methylenephosphonic acid) (DTPMPA or DTMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N, N-diacetic acid (α-ALDA), serine-N, N-diacetic acid (SEDA), isoserineN, N-diacetic acid (ISDA), phenylalanine-N, N-diacetic acid (PHDA), anthranilic acid-N, N-diacetic acid (ANDA), sulfanilic acid-N, N-diacetic acid (SLDA), taurine-N, N-diacetic acid (TUDA) and sulfomethyl-N, N-diacetic acid (SMDA), N-(2-hydroxyethyl)-ethylidenediamine-N,N′,N′-triacetate (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 2009/102854 and U.S. Pat. No. 5,977,053.

The protease variants of the invention may also be formulated into a dish wash composition, preferably an automatic dish wash composition (ADW), comprising:

a) at least 0.01 mg of active protease variant according to the invention, and

b) 10-50 wt % builder preferably selected from citric acid, methylglycine-N,N-diacetic acid (MGDA) and/or glutamic acid-N,N-diacetic acid (GLDA) and mixtures thereof, and

c) at least one bleach component.

### Bleaching Systems

The detergent may contain 0-50% by weight, such as about 0.1% to about 25%, of a bleaching system. Bleach systems remove discolor often by oxidation, and many bleaches also have strong bactericidal properties, and are used for disinfecting and sterilizing. Any bleaching system known in the art for use in laundry detergents may be utilized. Suitable bleaching system components include bleaching catalysts, photobleaches, bleach activators, sources of hydrogen peroxide such as sodium percarbonate and sodium perborates, preformed peracids and mixtures thereof. Suitable preformed peracids include, but are not limited to, peroxycarboxylic acids and salts, percarbonic acids and salts, perimidic acids and salts, peroxymonosulfuric acids and salts, for example, Oxone®, and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator.

The term bleach activator is meant herein as a compound which reacts with peroxygen bleach like hydrogen peroxide to form a peracid. The peracid thus formed constitutes the activated bleach. Suitable bleach activators to be used herein include those belonging to the class of esters amides, imides or anhydrides. Suitable examples are tetracetylethylene diamine (TAED), sodium 4-[(3,5,5-trimethylhexanoyl)oxy]benzene sulfonate (ISONOBS), diperoxy dodecanoic acid, 4-(dodecanoyloxy) benzenesulfonate (LOBS), 4-(decanoyloxy)benzenesulfonate, 4-(decanoyloxy)benzoate (DOBS), 4-(nonanoyloxy)-benzenesulfonate (NOBS), and/or those disclosed in WO 98/17767. A particular family of bleach activators of interest was disclosed in EP 624154 and particularly preferred in that family is acetyl triethyl citrate (ATC). ATC or a short chain triglyceride like triacetin has the advantage that it is environmentally friendly as it eventually degrades into citric acid and alcohol. Furthermore, acetyl triethyl citrate and triacetin have good hydrolytic stability in the product upon storage and are efficient bleach activators. Finally, ATC provides a good building capacity to the laundry additive. Alternatively, the bleaching system may comprise peroxyacids of, for example, the amide, imide, or sulfone type. The bleaching system may also comprise peracids such as 6-(phthalimido)peroxyhexanoic acid (PAP). The bleaching system may also include a bleach catalyst or a booster.

Some non-limiting examples of bleach catalysts that may be used in the compositions of the present invention include manganese oxalate, manganese acetate, manganese-collagen, cobalt-amine catalysts and manganese triazacyclononane (MnTACN) catalysts; particularly preferred are complexes of manganese with 1,4,7-trimethyl-1,4,7-triazacyclononane (Me3-TACN) or 1,2,4,7-tetramethyl-1,4,7-triazacyclononane (Me4-TACN), in particular Me3-TACN, such as the dinuclear manganese complex [(Me3-TACN)Mn(O)3Mn(Me3-TACN)](PF6)2, and [2,2′,2″-nitrilotris(ethane-1,2-diylazanylylidene-κN-methanylylidene)triphenolato-κ3O]manganese(III). The bleach catalysts may also be other metal compounds, such as iron or cobalt complexes.

In some embodiments, the bleach component may be an organic catalyst selected from the group consisting of organic catalysts having the following formula:

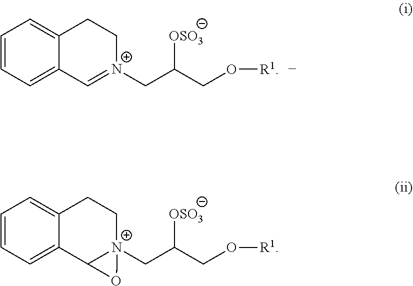

(iii) and mixtures thereof; wherein each R1 is independently a branched alkyl group containing from 9 to 24 carbons or linear alkyl group containing from 11 to 24 carbons, preferably each R1 is independently a branched alkyl group containing from 9 to 18 carbons or linear alkyl group containing from 11 to 18 carbons, more preferably each R1 is independently selected from the group consisting of 2-propylheptyl, 2-butyloctyl, 2-pentylnonyl, 2-hexyldecyl, n-dodecyl, n-tetradecyl, n-hexadecyl, n-octadecyl, iso-nonyl, iso-decyl, iso-tridecyl and iso-pentadecyl. Other exemplary bleaching systems are described, e.g., in WO 2007/087258, WO 2007/087244, WO 2007/087259 and WO 2007/087242. Suitable photobleaches may for example be sulfonated zinc phthalocyanine.

### Hydrotropes

A hydrotrope is a compound that solubilizes hydrophobic compounds in aqueous solutions (or oppositely, polar substances in a non-polar environment). Typically, hydrotropes have both hydrophilic and hydrophobic characters (so-called amphiphilic properties as known from surfactants); however, the molecular structures of hydrotropes generally do not favour spontaneous self-aggregation, see, e.g., review by Hodgdon and Kaler, 2007, Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behaviour, stability, and colloidal properties of systems containing substances of polar and non-polar character, including mixtures of water, oil, surfactants, and polymers. Hydrotropes are classically used across industries from pharma, personal care and food to technical applications. Use of hydrotropes in detergent compositions allows for example more concentrated formulations of surfactants (as in the process of compacting liquid detergents by removing water) without inducing undesired phenomena such as phase separation or high viscosity.

The detergent may contain 0-5% by weight, such as about 0.5 to about 5%, or about 3% to about 5%, of a hydrotrope. Any hydrotrope known in the art for use in detergents may be utilized. Non-limiting examples of hydrotropes include sodium benzene sulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Polymers

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

### Fabric Hueing Agents

The detergent compositions of the present invention may also include fabric hueing agents such as dyes or pigments, which when formulated in detergent compositions can deposit onto a fabric when the fabric is contacted with a wash liquor comprising the detergent compositions and thus altering the tint of the fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO 2005/003274, WO 2005/003275, WO 2005/003276 and EP 1876226 (hereby incorporated by reference). The detergent composition preferably comprises from about 0.00003 wt. % to about 0.2 wt. %, from about 0.00008 wt. % to about 0.05 wt. %, or even from about 0.0001 wt. % to about 0.04 wt. % fabric hueing agent. The composition may comprise from 0.0001 wt % to 0.2 wt. % fabric hueing agent, this may be especially preferred when the composition is in the form of a unit dose pouch. Suitable hueing agents are also disclosed in, e.g., WO 2007/087257 and WO 2007/087243.

### Additional Enzymes

The detergent composition may comprise one or more additional enzymes such as an amylase, an arabinase, a carbohydrase, a cellulase (e.g., endoglucanase), a cutinase, a deoxyribonuclease, a galactanase, a haloperoxygenase, a lipase, a mannanase, an oxidase, e.g., a laccase and/or peroxidase, a pectinase, a pectin lyase, an additional protease, a xylanase, a xanthanase, a xyloglucanase or an oxidoreductase.

When the composition comprises one or more additional enzymes, the additional enzyme is preferably an amylase and/or a lipase, in particular an amylase.

The properties of the selected enzyme(s) should be compatible with the selected detergent (e.g., pH-optimum, compatibility with other enzymatic and non-enzymatic ingredients, etc.).

### Proteases

The composition may, in addition to a protease variant of the invention, comprise one or more additional proteases including those of bacterial, fungal, plant, viral or animal origin. Proteases of microbial origin are preferred. The protease may be an alkaline protease, such as a serine protease or a metalloprotease. A serine protease may for example be of the S1 family, such as trypsin, or the S8 family such as subtilisin. A metalloprotease may for example be a thermolysin from, e.g., family M4 or another metalloprotease such as those from M5, M7 or M8 families.

Examples of metalloproteases are the neutral metalloproteases as described in WO 2007/044993 (Genencor Int.) such as those derived from Bacillus amyloliquefaciens.

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Blaze®, Blaze Evity® 100T, Blaze Evity® 125T, Blaze Evity® 150T, Neutrase®, Everlase®, Esperase®, Progress® Uno and Progress® Excel (Novozymes A/S), those sold under the tradenames Maxatase®, Maxacal®, Maxapem®, Purafect®, Purafect® Ox, Purafect® OxP, Purafect Prime®, Puramax®, FN2®, FN3®, FN4®, Excellase®, Excellenz P1000™, Excellenz P1250™, Eraser®, Preferenz® P100, Preferenz® P110, Effectenz P1000™, Effectenz P1050™, Effectenz P2000™, Purafast®, Properase®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

### Lipases and Cutinases

Suitable lipases and cutinases include those of bacterial or fungal origin. Chemically modified or protein engineered mutant enzymes are included. Examples include lipase from Thermomyces, e.g., from T. lanuginosus (previously named Humicola lanuginosa) as described in EP 258068 and EP 305216, cutinase from Humicola, e.g., H. insolens (WO 96/13580), lipase from strains of Pseudomonas (some of these now renamed to Burkholderia), e.g., P. alcaligenes or P. pseudoalcaligenes (EP 218272), P. cepacia (EP 331376), P. sp. strain SD705 (WO 95/06720 & WO 96/27002), P. wisconsinensis (WO 96/12012), GDSL-type Streptomyces lipases (WO 2010/065455), cutinase from Magnaporthe grisea (WO 2010/107560), cutinase from Pseudomonas mendocina (U.S. Pat. No. 5,389,536), lipase from Thermobifida fusca (WO 2011/084412), Geobacillus stearothermophilus lipase (WO 2011/084417), lipase from Bacillus subtilis (WO 2011/084599), and lipase from Streptomyces griseus (WO 2011/150157) and S. pristinaespiralis (WO 2012/137147).

Other examples are lipase variants such as those described in EP 407225, WO 92/05249, WO 94/01541, WO 94/25578, WO 95/14783, WO 95/30744, WO 95/35381, WO 95/22615, WO 96/00292, WO 97/04079, WO 97/07202, WO 00/34450, WO 00/60063, WO 01/92502, WO 2007/87508 and WO 2009/109500.

Preferred commercial lipase products include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g., acyltransferases with homology to Candida antarctica lipase A (WO 2010/111143), acyltransferase from Mycobacterium smegmatis (WO 2005/056782), perhydrolases from the CE 7 family (WO 2009/067279), and variants of the M. smegmatis perhydrolase, in particular the S54V variant used in the commercial product Gentle Power Bleach from Huntsman Textile Effects Pte Ltd (WO 2010/100028).

### Amylases

Suitable amylases which can be used together with the protease variant of the invention may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO:2 in WO 95/10603 or variants having 90% sequence identity to SEQ ID NO:3 thereof. Preferred variants are described in WO 94/02597, WO 94/18314, WO 97/43424 and SEQ ID NO: 4 of WO 99/19467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO:6 in WO 2002/10355 or variants thereof having 90% sequence identity to SEQ ID NO:6. Preferred variants of SEQ ID NO:6 are those having a deletion in positions 181 and 182 and a substitution in position 193.

Other amylases which are suitable are hybrid alpha-amylases comprising residues 1-33 of the alpha-amylase derived from B. amyloliquefaciens shown in SEQ ID NO:6 of WO 2006/066594 and residues 36-483 of the B. licheniformis alpha-amylase shown in SEQ ID NO:4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO:6 of WO 2006/066594 and residues 36-483 of SEQ ID NO: 4 are those having the substitutions:

M197T;

H156Y+A181T+N190F+A209V+Q264S; or

G48A+T49I+G107A+H156Y+A181T+N190F+1201F+A209V+Q264S.

Other suitable amylases are amylases having the sequence of SEQ ID NO:6 in WO 99/19467 or variants thereof having 90% sequence identity to SEQ ID NO:6. Preferred variants of SEQ ID NO:6 are those having a substitution, a deletion or an insertion in one or more of the following positions: R181, G182, H183, G184, N195, I206, E212, E216 and K269. Particularly preferred amylases are those having deletion in positions R181 and G182, or positions H183 and G184.

Additional amylases which can be used are those having SEQ ID NO:1, SEQ ID NO:3, SEQ ID NO:2 or SEQ ID NO:7 of WO 96/23873 or variants thereof having 90% sequence identity to SEQ ID NO:1, SEQ ID NO:2, SEQ ID NO:3 or SEQ ID NO:7. Preferred variants of SEQ ID NO:1, SEQ ID NO:2, SEQ ID NO:3 or SEQ ID NO:7 are those having a substitution, a deletion or an insertion in one or more of the following positions: 140, 181, 182, 183, 184, 195, 206, 212, 243, 260, 269, 304 and 476, using SEQ ID NO:2 of WO 96/23873 for numbering. More preferred variants are those having a deletion in two positions selected from 181, 182, 183 and 184, such as 181 and 182, 182 and 183, or positions 183 and 184. Most preferred amylase variants of SEQ ID NO:1, SEQ ID NO:2 or SEQ ID NO:7 are those having a deletion in positions 183 and 184 and a substitution in one or more of positions 140, 195, 206, 243, 260, 304 and 476.

Other amylases which can be used are amylases having SEQ ID NO:2 of WO 2008/153815, SEQ ID NO:10 in WO 01/66712 or variants thereof having 90% sequence identity to SEQ ID NO:2 of WO 2008/153815 or 90% sequence identity to SEQ ID NO:10 in WO 01/66712. Preferred variants of SEQ ID NO:10 in WO 01/66712 are those having a substitution, a deletion or an insertion in one of more of the following positions: 176, 177, 178, 179, 190, 201, 207, 211 and 264.

Further suitable amylases are amylases having SEQ ID NO:2 of WO 2009/061380 or variants having 90% sequence identity to SEQ ID NO:2 thereof. Preferred variants of SEQ ID NO:2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO:2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO:2 are those having the substitutions:

N128C+K178L+T182G+Y305R+G475K;

N128C+K178L+T182G+F202Y+Y305R+D319T+G475K;

S125A+N128C+K178L+T182G+Y305R+G475K; or

S125A+N128C+T131I+T165I+K178L+T182G+Y305R+G475K,

wherein the variants are C-terminally truncated and optionally further comprise a substitution at position 243 and/or a deletion at position 180 and/or position 181.

Further suitable amylases are amylases having SEQ ID NO:1 of WO 2013/184577 or variants having 90% sequence identity to SEQ ID NO:1 thereof. Preferred variants of SEQ ID NO:1 are those having a substitution, a deletion or an insertion in one of more of the following positions: K176, R178, G179, T180, G181, E187, N192, M199, 1203, S241, R458, T459, D460, G476 and G477. More preferred variants of SEQ ID NO:1 are those having the substitution in one of more of the following positions: K176L, E187P, N192FYH, M199L, I203YF, S241QADN, R458N, T459S, D460T, G476K and G477K and/or a deletion in position R178 and/or S179 or of T180 and/or G181. Most preferred amylase variants of SEQ ID NO:1 comprise the substitutions:

E187P+I203Y+G476K

E187P+I203Y+R458N+T459S+D460T+G476K

and optionally further comprise a substitution at position 241 and/or a deletion at position 178 and/or position 179.

Further suitable amylases are amylases having SEQ ID NO:1 of WO 2010/104675 or variants having 90% sequence identity to SEQ ID NO:1 thereof. Preferred variants of SEQ ID NO:1 are those having a substitution, a deletion or an insertion in one of more of the following positions: N21, D97, V128 K177, R179, S180, I181, G182, M200, L204, E242, G477 and G478.

More preferred variants of SEQ ID NO:1 are those having the substitution in one of more of the following positions: N21D, D97N, V128I K177L, M200L, L204YF, E242QA, G477K and G478K and/or a deletion in position R179 and/or S180 or of 1181 and/or G182. Most preferred amylase variants of SEQ ID NO:1 comprise the substitutions N21D+D97N+V128I, and optionally further comprise a substitution at position 200 and/or a deletion at position 180 and/or position 181.

Other suitable amylases are the alpha-amylase having SEQ ID NO:12 in WO 01/66712 or a variant having at least 90% sequence identity to SEQ ID NO:12. Preferred amylase variants are those having a substitution, a deletion or an insertion in one of more of the following positions of SEQ ID NO:12 in WO 01/66712: R28, R118, N174, R181, G182, D183, G184, G186, W189, N195, M202, Y298, N299, K302, S303, N306, R310, N314, R320, H324, E345, Y396, R400, W439, R444, N445, K446, Q449, R458, N471, N484. Particularly preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO 2011/098531, WO 2013/001078 and WO 2013/001087. Commercially available amylases include DuramylT™, Termamyl™, Fungamyl™, Stainzyme™, Stainzyme Plus™, Natalase™, Liquozyme X, BAN™, Amplify® and Amplify® Prime (from Novozymes A/S), and Rapidase™, Purastar™/Effectenz™, Powerase, Preferenz S1000, Preferenz S100 and Preferenz S110 (from Genencor International Inc./DuPont).

One preferred amylase is a variant of the amylase having SEQ ID NO:13 in WO 2016/180748 with the alterations H1*+N54S+V56T+K72R+G109A+F113Q+R116Q+W167F+Q172G+A174S+G182*+D183*+G184T+N195F+V206L+K391A+P473R+G476K.

Another preferred amylase is a variant of the amylase having SEQ ID NO:1 in WO 2013/001078 with the alterations D183*+G184*+W140Y+N195F+V206Y+Y243F+E260G+G304R+G476K.

Another preferred amylase is a variant of the amylase having SEQ ID NO:1 in WO 2018/141707 with the alterations H1*+G7A+G109A+W140Y+G182*+D183*+N195F+V206Y+Y243F+E260G+N280S+G304R+E391A+G476K.

A further preferred amylase is a variant of the amylase having SEQ ID NO:1 in WO 2017/191160 with the alterations L202M+T246V.

### Deoxyribonucleases (DNases)

Suitable deoxyribonucleases (DNases) are any enzyme that catalyzes the hydrolytic cleavage of phosphodiester linkages in the DNA backbone, thus degrading DNA. A DNase which is obtainable from a bacterium is preferred, in particular a DNase which is obtainable from a species of Bacillus is preferred; in particular a DNase which is obtainable from Bacillus subtilis or Bacillus licheniformis is preferred. Examples of such DNases are described in WO 2011/098579 and WO 2014/087011.

### Oxidoreductases

In one embodiment, the composition may comprise an oxidoreductase, which are enzymes that catalyze reduction-oxidation reactions. A preferred oxidoreductase is a superoxide dismutase.

### Peroxidases/Oxidases

Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257.

Commercially available peroxidases include Guardzyme™ (Novozymes A/S).

### Adjunct Materials

Any detergent components known in the art for use in laundry detergents may also be utilized. Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in laundry detergents may be utilized. The choice of such ingredients is well within the skill of the artisan.

Dispersants: The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant Science Series, volume 71, Marcel Dekker, Inc., 1997.

Dye Transfer Inhibiting Agents: The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

Fluorescent whitening agent: The detergent compositions of the present invention will preferably also contain additional components that may tint articles being cleaned, such as fluorescent whitening agent or optical brighteners. Where present the brightener is preferably at a level of about 0.01% to about 05%. Any fluorescent whitening agent suitable for use in a laundry detergent composition may be used in the composition of the present invention. The most commonly used fluorescent whitening agents are those belonging to the classes of diaminostilbene-sulphonic acid derivatives, diarylpyrazoline derivatives and bisphenyl-distyryl derivatives. Examples of the diaminostilbene-sulphonic acid derivative type of fluorescent whitening agents include the sodium salts of: 4,4′-bis-(2-diethanolamino-4-anilino-s-triazin-6-ylamino) stilbene-2,2′-disulphonate; 4,4′-bis-(2,4-dianilino-s-triazin-6-ylamino) stilbene-2.2′-disulphonate; 4,4′-bis-(2-anilino-4(N-methyl-N-2-hydroxy-ethylamino)-s-triazin-6-ylamino) stilbene-2,2′-disulphonate, 4,4′-bis-(4-phenyl-2,1,3-triazol-2-yl)stilbene-2,2′-disulphonate; 4,4′-bis-(2-anilino-4(1-methyl-2-hydroxy-ethylamino)-s-triazin-6-ylamino) stilbene-2,2′-disulphonate and 2-(stilbyl-4″-naptho-1,2′:4,5)-1,2,3-trizole-2″-sulphonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4 anilino-s-triazin-6-ylamino) stilbene disulphonate. Tinopal CBS is the disodium salt of 2,2′-bis-(phenyl-styryl) disulphonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins. Suitable fluorescent brightener levels include lower levels of from about 0.01, from 0.05, from about 0.1 or even from about 0.2 wt. % to upper levels of 0.5 or even 0.75 wt. %.

Soil release polymers: The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers is amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore, random graft co-polymers are suitable soil release polymers Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 03/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

Anti-redeposition agents: The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

Other suitable adjunct materials include, but are not limited to, anti-shrink agents, anti-wrinkling agents, bactericides, binders, carriers, dyes, enzyme stabilizers, fabric softeners, fillers, foam regulators, perfumes, pigments, sod suppressors, solvents, and structurants for liquid detergents and/or structure elasticizing agents.

### Formulation of Detergent Products

The detergent enzyme(s), i.e., a protease variant of the invention and optionally one or more additional enzymes, may be included in a detergent composition by adding separate additives containing one or more enzymes, or by adding a combined additive comprising all of these enzymes. A detergent additive comprising one or more enzymes can be formulated, for example, as a granulate, liquid, slurry, etc. Preferred detergent additive formulations include granulates, in particular non-dusting granulates, liquids, in particular stabilized liquids, or slurries.

The detergent composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid. There are a number of detergent formulation forms such as layers (same or different phases), pouches, as well as forms for machine dosing unit.

Pouches can be configured as single or multiple compartments. It can be of any form, shape and material which is suitable for hold the composition, e.g., without allowing the release of the composition from the pouch prior to water contact. The pouch is made from water soluble film which encloses an inner volume. The inner volume can be divided into compartments of the pouch. Preferred films are polymeric materials, preferably polymers which are formed into a film or sheet. Preferred polymers, copolymers or derivates thereof are selected from polyacrylates, and water-soluble acrylate copolymers, methyl cellulose, carboxy methyl cellulose, sodium dextrin, ethyl cellulose, hydroxyethyl cellulose, hydroxypropyl methyl cellulose, maltodextrin, polymethacrylates, most preferably polyvinyl alcohol copolymers and hydroxypropyl methyl cellulose (HPMC). Preferably the level of polymer in the film for example PVA is at least about 60%. The preferred average molecular weight will typically be about 20,000 to about 150,000. Films can also be of blend compositions comprising hydrolytically degradable and water-soluble polymer blends such as polylactide and polyvinyl alcohol (known under the Trade reference M8630 as sold by Chris Craft In. Prod. of Gary, Ind., US) plus plasticizers like glycerol, ethylene glycerol, propylene glycol, sorbitol and mixtures thereof. The pouches can comprise a solid laundry detergent composition or part components and/or a liquid cleaning composition or part components separated by the water-soluble film. The compartment for liquid components can be different in composition than compartments containing solids. See, e.g., US 2009/0011970.

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent which is not unit dosed may be aqueous, typically containing at least 20% by weight and up to 95% water, such as up to about 70% water, up to about 65% water, up to about 55% water, up to about 45% water, or up to about 35% water. Concentrated liquid detergents may have lower water contents, for example not more than about 30% or not more than about 20%, e.g. in the range of about 1% to about 20%, such as from about 2% to about 15%. Other types of liquids, including without limitation, alkanols, amines, diols, ethers and polyols may be included in an aqueous liquid or gel. An aqueous liquid or gel detergent may contain from 0-30% organic solvent. A liquid or gel detergent may be non-aqueous.

Liquid detergent compositions may be formulated to have a moderate pH of e.g. from about 6 to about 10, such as about pH 7, about pH 8 or about pH 9, or they may be formulated to have a higher pH of e.g. from about 10 to about 12, such as about pH 10, about pH 11 or about pH 12.

Unless indicated otherwise, the term “liquid” as used herein should be understood to encompass any kind of liquid detergent composition, for example concentrated liquids, gels, or the liquid or gel part of e.g. a pouch with one or more compartments.

### Laundry Soap Bars

The enzymes of the invention may be added to laundry soap bars and used for hand washing laundry, fabrics and/or textiles. The term laundry soap bar includes laundry bars, soap bars, combo bars, syndet bars and detergent bars. The types of bar usually differ in the type of surfactant they contain, and the term laundry soap bar includes those containing soaps from fatty acids and/or synthetic soaps. The laundry soap bar has a physical form which is solid and thus not a liquid, gel or powder at room temperature.

The laundry soap bar may contain one or more additional enzymes, protease inhibitors such as peptide aldehydes (or hydrosulfite adduct or hemiacetal adduct), boric acid, borate, borax and/or phenylboronic acid derivatives such as 4-formylphenylboronic acid, one or more soaps or synthetic surfactants, polyols such as glycerine, pH controlling compounds such as fatty acids, citric acid, acetic acid and/or formic acid, and/or a salt of a monovalent cation and an organic anion, wherein the monovalent cation may be for example Na⁺, K⁺ or NH₄⁺, and the organic anion may be for example formate, acetate, citrate, or lactate such that the salt of a monovalent cation and an organic anion may be, for example, sodium formate.

The laundry soap bar may also contain complexing agents such as EDTA and HEDP, perfumes and/or different type of fillers, surfactants, e.g., anionic synthetic surfactants, builders, polymeric soil release agents, detergent chelators, stabilizing agents, fillers, dyes, colorants, dye transfer inhibitors, alkoxylated polycarbonates, suds suppressers, structurants, binders, leaching agents, bleaching activators, clay soil removal agents, anti-redeposition agents, polymeric dispersing agents, brighteners, fabric softeners, perfumes and/or other compounds known in the art.

The laundry soap bar may be processed in conventional laundry soap bar making equipment such as but not limited to mixers, plodders, e.g., a two-stage vacuum plodder, extruders, cutters, logo-stampers, cooling tunnels and wrappers. A premix containing a soap, the enzyme of the invention, optionally one or more additional enzymes, a protease inhibitor, and a salt of a monovalent cation and an organic anion may be prepared, and the mixture is then plodded. The enzyme and optional additional enzymes may be added at the same time as the protease inhibitor for example in liquid form. Besides the mixing step and the plodding step, the process may further comprise the steps of milling, extruding, cutting, stamping, cooling and/or wrapping.

### Granular Detergent Formulations

Enzymes in the form of granules, comprising an enzyme-containing core and optionally one or more coatings, are commonly used in granular (powder) detergents. Various methods for preparing the core are well-known in the art and include, for example, a) spray drying of a liquid enzyme-containing solution, b) production of layered products with an enzyme coated as a layer around a pre-formed inert core particle, e.g. using a fluid bed apparatus, c) absorbing an enzyme onto and/or into the surface of a pre-formed core, d) extrusion of an enzyme-containing paste, e) suspending an enzyme-containing powder in molten wax and atomization to result in prilled products, f) mixer granulation by adding an enzyme-containing liquid to a dry powder composition of granulation components, g) size reduction of enzyme-containing cores by milling or crushing of larger particles, pellets, etc., and h) fluid bed granulation. The enzyme-containing cores may be dried, e.g. using a fluid bed drier or other known methods, for drying granules in the feed or enzyme industry, to result in a water content of typically 0.1-10% w/w water.

The enzyme-containing cores are optionally provided with a coating to improve storage stability and/or to reduce dust formation. One type of coating that is often used for enzyme granulates for detergents is a salt coating, typically an inorganic salt coating, which may e.g. be applied as a solution of the salt using a fluid bed. Other coating materials that may be used are, for example, polyethylene glycol (PEG), methyl hydroxy-propyl cellulose (MHPC) and polyvinyl alcohol (PVA). The granules may contain more than one coating, for example a salt coating followed by an additional coating of a material such as PEG, MHPC or PVA.

For further information on enzyme granules and production thereof, see WO 2013/007594 as well as e.g. WO 2009/092699, EP 1705241, EP 1382668, WO 2007/001262, U.S. Pat. No. 6,472,364, WO 2004/074419 and WO 2009/102854.

### Uses and Cleaning Methods

The present invention is also directed to methods for using the protease variants according to the invention or compositions thereof in laundering of textile and fabrics, such as household laundry washing and industrial laundry washing.

The invention is also directed to methods for using the variants according to the invention or compositions thereof in cleaning hard surfaces such as floors, tables, walls, roofs etc. as well as surfaces of hard objects such as cars (car wash) and dishes (dish wash).

The protease variants of the present invention may be added to and thus become a component of a detergent composition. Thus, one aspect of the invention relates to the use of a protease variant in a cleaning process such as laundering and/or hard surface cleaning.

A detergent composition of the present invention may be formulated, for example, as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations.

The cleaning process or the textile care process may for example be a laundry process, a dishwashing process or cleaning of hard surfaces such as bathroom tiles, floors, tabletops, drains, sinks and washbasins. Laundry processes can for example be household laundering but may also be industrial laundering. Furthermore, the invention relates to a process for laundering of fabrics and/or garments, where the process comprises treating fabrics with a washing solution containing a detergent composition and at least one protease variant of the invention. The cleaning process or a textile care process can for example be carried out in a machine washing or manually. The washing solution can for example be an aqueous washing solution containing a detergent composition.

The last few years there has been an increasing interest in replacing components in detergents that are derived from petrochemicals with renewable biological components such as enzymes and polypeptides without compromising the wash performance. When the components of detergent compositions change, new enzyme activities or new enzymes having alternative and/or improved properties compared to the previously used detergent enzymes such as proteases, lipases and amylases may be needed to achieve a similar or improved wash performance when compared to the traditional detergent compositions.

The invention further concerns the use of protease variants of the invention in a proteinaceous stain removing process. The proteinaceous stains may be stains such as food stains, e.g., baby food, cocoa, egg or milk, or other stains such as sebum, blood, ink or grass, or a combination hereof.

### Washing Method

The present invention provides a method of cleaning a fabric, dishware or a hard surface with a detergent composition comprising a protease variant of the invention.

The method of cleaning comprises contacting an object with a detergent composition comprising a protease variant of the invention under conditions suitable for cleaning the object. In a preferred embodiment the detergent composition is used in a laundry or a dish wash process.

Another embodiment relates to a method for removing stains from fabric or dishware which comprises contacting the fabric or dishware with a composition comprising a protease of the invention under conditions suitable for cleaning the object. In the method of cleaning of the invention, the object being cleaned may be any suitable object such as a textile or a hard surface such as dishware or a floor, table, wall, etc.

Also contemplated are compositions and methods of treating fabrics (e.g., to desize a textile) using one or more of the protease variants of the invention. The protease can be used in any fabric-treating method which is well known in the art (see, e.g., U.S. Pat. No. 6,077,316). For example, in one aspect, the feel and appearance of a fabric is improved by a method comprising contacting the fabric with a protease in a solution. In one aspect, the fabric is treated with the solution under pressure.

The detergent compositions of the present invention are suited for use in laundry and hard surface applications, including dish wash. Accordingly, the present invention includes a method for laundering a fabric or washing dishware, comprising contacting the fabric/dishware to be cleaned with a solution comprising the detergent composition according to the invention. The fabric may comprise any fabric capable of being laundered in normal consumer use conditions. The dishware may comprise any dishware such as crockery, cutlery, ceramics, plastics such as melamine, metals, china, glass and acrylics. The solution preferably has a pH from about 5.5 to about 11.5. The compositions may be employed at concentrations from about 100 ppm, preferably 500 ppm to about 15,000 ppm in solution. The water temperatures typically range from about 5° C. to about 95° C., including about 10° C., about 15° C., about 20° C., about 25° C., about 30° C., about 35° C., about 40° C., about 45° C., about 50° C., about 55° C., about 60° C., about 65° C., about 70° C., about 75° C., about 80° C., about 85° C. and about 90° C. The water to fabric ratio is typically from about 1:1 to about 30:1.

The enzyme(s) of the detergent composition of the invention may be stabilized using conventional stabilizing agents and protease inhibitors, e.g., a polyol such as propylene glycol or glycerol, a sugar or sugar alcohol, different salts such as NaCl; KCl; lactic acid, formic acid, boric acid, or a boric acid derivative, e.g., an aromatic borate ester, or a phenyl boronic acid derivative such as 4-formylphenyl boronic acid, or a peptide aldehyde such as di-, tri- or tetrapeptide aldehydes or aldehyde analogues (either of the form B1-B0-R wherein, R is H, CH3, CX3, CHX2, or CH2X (X=halogen), B0 is a single amino acid residue (preferably with an optionally substituted aliphatic or aromatic side chain); and B1 consists of one or more amino acid residues (preferably one, two or three), optionally comprising an N-terminal protection group, or as described in WO 2009/118375, WO 98/13459) or a protease inhibitor of the protein type such as RASI, BASI, WASI (bifunctional alpha-amylase/subtilisin inhibitors of rice, barley and wheat) or Cl2 or SSI. The composition may be formulated as described in, e.g., WO 92/19709, WO 92/19708 and U.S. Pat. No. 6,472,364. In some embodiments, the enzymes employed herein are stabilized by the presence of water-soluble sources of zinc (II), calcium (II) and/or magnesium (II) ions in the finished compositions that provide such ions to the enzymes, as well as other metal ions (e.g., barium (II), scandium (II), iron (II), manganese (II), aluminum (III), Tin (II), cobalt (II), copper (II), Nickel (II), and oxovanadium (IV)).

The detergent compositions provided herein are typically formulated such that, during use in aqueous cleaning operations, the wash water has a pH of from about 5.0 to about 12.5, such as from about 5.0 to about 11.5, or from about 6.0 to about 10.5. In some embodiments, granular or liquid laundry products are formulated to have a pH from about 6 to about 8. Techniques for controlling pH at recommended usage levels include the use of buffers, alkalis, acids, etc., and are well known to those skilled in the art.

The present invention is further described by the following examples that should not be construed as limiting the scope of the invention.

### EXAMPLES

### Preparation and Purification of Polypeptides

Mutation and introduction of expression cassettes into Bacillus subtilis was performed by standard methods known in the art. All DNA manipulations were performed by PCR (e.g., as described by Sambrook et al., 2001) using standard methods known to the skilled person. Recombinant B. subtilis constructs encoding protease polypeptides were inoculated into and cultivated in a complex medium (TBgly) under antibiotic selection for 24 h at 37° C. Shake flasks containing a rich media (PS-1: 100 g/L sucrose (Danisco cat. no. 109-0429), 40 g/L crust soy (soybean flour), 10 g/L Na₂HPO₄ 12H₂O (Merck cat. no. 106579), 0.1 ml/L Dowfax63N10 (Dow) were inoculated in a ratio of 1:100 with the overnight culture. Shake flask cultivation was performed for 4 days at 30° C. shaking at 270 rpm.

Purification of culture supernatants was performed as follows: The culture broth is centrifuged at 26,000×g for 20 minutes and the supernatant is carefully decanted from the precipitate. The supernatant is filtered through a Nalgene 0.2 μm filtration unit in order to remove the remains of the host cells. The pH in the 0.2 μm filtrate is adjusted to pH 8 with 3 M Tris base and the pH-adjusted filtrate is applied to a MEP Hypercel column (Pall Corporation) equilibrated in 20 mM Tris/HCl, 1 mM CaCl₂), pH 8.0. After washing the column with the equilibration buffer, the column is step-eluted with 20 mM CH₃COOH/NaOH, 1 mM CaCl₂), pH 4.5. Fractions from the column are analyzed for protease activity using the Suc-AAPF-pNA assay at pH 9 and peak fractions are pooled. The pH of the pool from the MEP Hypercel column is adjusted to pH 6 with 20% (v/v) CH₃COOH or 3 M Tris base and the pH-adjusted pool is diluted with deionized water to the same conductivity as 20 mM MES/NaOH, 2 mM CaCl₂), pH 6.0. The diluted pool is applied to an SP-Sepharose® Fast Flow column (GE Healthcare) equilibrated in 20 mM MES/NaOH, 2 mM CaCl₂), pH 6.0. After washing the column with the equilibration buffer, the protease variant is eluted with a linear NaCl gradient (0→0.5 M) in the same buffer over five column volumes. Fractions from the column are analyzed for protease activity using the Suc-AAPF-pNA assay at pH 9 and active fractions are analyzed by SDS-PAGE. Fractions in which only one band is observed on the Coomassie stained SDS-PAGE gel are pooled as the purified preparation and used for further experiments.

### Protease Activity Assay

Proteolytic activity can be determined by a method employing the Suc-AAPF-pNA substrate. Suc-AAPF-pNA is an abbreviation for N-Succinyl-Alanine-Alanine-Proline-Phenylalanine-p-Nitroanilide, and it is a blocked peptide which can be cleaved by endo-proteases. Following proteolytic cleavage, a free pNA molecule having a yellow color is liberated and can be measured by visible spectrophotometry at wavelength 405 nm. The Suc-AAPF-PNA substrate is manufactured by Bachem (cat. no. L1400, dissolved in DMSO).

The protease sample to be analyzed is diluted in residual activity buffer (100 mM Tris, pH 8.6). The assay is performed by transferring 30 μl of diluted enzyme samples to 96 well microtiter plate and adding 70 μl substrate working solution (0.72 mg/ml in 100 mM Tris, pH 8.6). The solution is mixed at room temperature and absorption is measured every 20 sec. over 5 minutes at OD 405 nm.

The slope (absorbance per minute) of the time dependent absorption-curve is directly proportional to the activity of the protease in question under the given set of conditions. The protease sample should be diluted to a level where the slope is linear.

### Example 1. Improved Solubility of Protease Variants

### Definitions

Fermentation broth:
A_{CB}=the full protease activity (including crystallized protease) in the fermentation broth.
A_{CB SUP}=the dissolved protease activity in the fermentation broth.
A_{CB PEL}=the pellet protease activity (including crystallized protease) in the fermentation broth.
A_{INIT}=The percentage of dissolved protease activity in the fermentation broth.

### Protease Crystal Dissolution

A_{FULL}=the full protease activity (including crystallized protease) in the diluted fermentation broth.
A_{EXP}=the expected full protease activity of the diluted fermentation broth based on the protease activity of the fermentation broth and the dilution factor:

$${A}_{E X P} = \frac{weight of fermentation broth}{weight of diluted fermentation broth} * {A}_{C B}$$

A_{SUP}=the dissolved protease activity in the diluted fermentation broth.
A_{CORR}=the difference between the measured activity in the fermentation broth and expected full protease activity in the diluted fermentation broth in percent:

$${A}_{C O R R} = \frac{{A}_{F U L L} - {A}_{E X P}}{{A}_{E X P}} * 100 \%$$

A_{DISS}=The dissolved fraction of protease in the diluted fermentation broth in percent:

$${A}_{DISS} = \frac{{A}_{S U P}}{{A}_{E X P}} * 100 \%$$

### Materials and Methods:

### Initial Dissolved Protease Activity in Fermentation Broth:

Fermentation broths from host cells expressing protease variants with and without a substitution at a position corresponding to position A215 of SEQ ID NO:1 were harvested and analyzed for protease crystals. The presence of protease crystals was confirmed by light microscopy (Olympus BX51) and X-Ray Powder Diffraction (XRPD, PANalytical Empyrean) as described in Acta Cryst. (Frankaer, C. G., et. al. (2014). Acta Cryst. D70, 1115-1123).

Crystal solubility/formation was evaluated by investigating the dissolved protease activity (non-crystallized protease fraction), as initial dissolved protease activity in the fermentation broth (A_{INIT}). The following samples were collected:
A_{CB}: a full fermentation broth sample (including crystallized protease)
A_{CB SUP}: supernatant sample (dissolved protease)
A_{CB PEL}: a pellet sample from the culture broth containing crystallized protease.

The A_{CB SUP} and A_{CB PEL} were sampled by high-speed centrifugation (5 min, 10.000×RCF, 20° C.) and fractioned. The samples were subsequently analyzed by the protease activity assay described above. The protease activity in A_{CB} and A_{CB} sup were used to calculate A_{INIT} by:

$${A}_{INIT} = \frac{{A}_{CB SUP}}{{A}_{C B}} \cdot 100 \%$$

were A_{INIT} is the percentage of dissolved protease activity in the fermentation broth. The activity A_{CB PEL} sample were used as a control to evaluate the mass balance of the protease activity. Finally, A_{INIT} for the Protease_{+A215X} variant was normalized to A_{INIT} for the same protease without the A215X substitution (i.e., Protease_{-A215X}), yielding the difference in initial dissolved protease activity in the fermentation broth, given as fold increase:

$$P {A}_{INIT} = \frac{{Protease}_{+ A 215 X} {A}_{INIT}}{{Protease}_{- A 215 X} {A}_{INIT}}$$

where A215X denotes the particular substitution (e.g., A215K) introduced at a position corresponding to position A215 of SEQ ID NO:1 in the protease variant.

### Protease Crystal Dissolution:

To evaluate the crystal dissolution of the protease variants with and without a substitution at a position corresponding to position A215 of SEQ ID NO:1, the fermentation broths were diluted five times with H₂O, the pH level was adjusted to pH 4.5 with acetic acid (20%), and the conductivity was adjusted to 9 mS/cm with CaCl₂) (34%). The dissolution was conducted at a constant temperature of 20° C. and adequate mixing.

Immediately following dissolution, the experiment started. After a total of 15 min and 60 min, a full protease activity sample (A_{FULL}) and a supernatant protease activity sample (A_{SUP}) were collected. The A_{SUP} samples were sampled by high-speed centrifugation (5 min, 10.000×RCF, 20° C.) and the supernatants were decanted. All samples were subsequently analyzed by the protease activity assay described above.

For assessment of the crystal dissolution in the fermentation broths, the expected full protease activity (A_{EXP}) was calculated based on the protease activity of the fermentation broth and the dilution factor:

$${A}_{E X P} = \frac{weight of fermentation broth}{weight of flocculated fermentation broth} * {A}_{C B}$$

The A_{FULL} samples collected during the dissolution experiment were used to validate A_{EXP} by calculating the difference between A_{CB} and A_{FULL}, i.e., the difference between the measured activity in the fermentation broth and expected full protease activity in the fermentation broths:

$${A}_{C O R R} = \frac{{A}_{F U L L} - {A}_{E X P}}{{A}_{E X P}} * 100 \%$$

where A_{CORR} is given as a percentage.

The crystal dissolution was evaluated by calculating the fraction of the dissolved protease (A_{DISS}) at 60 min:

$${A}_{DISS} = \frac{{A}_{S U P}}{{A}_{E X P}} * 100 \%$$

Finally, A_{DISS} for the Protease_{+A215X} was normalized to A_{DISS} for the same protease without the A215X substitution, yielding the difference in protease crystal dissolution in the fermentation broths, given as a fold increase:

$$P {A}_{DISS} = \frac{{Protease}_{+ A 215 X} {A}_{DISS \_ T 60}}{{Protease}_{- A 215 X} {A}_{DISS \_ T 60}}$$

where A_{DISS_T60} denotes the value of A_{DISS} at 60 minutes.

### Results:

Table 1 shows the initial dissolved protease activity of A215X variants in culture broths. As can be seen, the A215K substitution gave rise to a 2.9 to 15.6-fold increase in initial dissolved protease activity measured in the fermentation broth across the five tested proteases. In addition, the substitutions A215Q and A215N provided a 5.1-fold and a 2.4-fold increase, respectively, in initial dissolved activity. The A215T substitution had a more subtle effect, providing a 1.1-fold increase, and no effect on initial dissolved activity was observed for the A215S variant. These data indicate that the degree of protease crystal formation is decreased by introduction of A215X substitutions.

TABLE 1
Initial dissolved activity of A215X protease variants evaluated
in culture broth. The dissolved activity is given as a fold
increase, normalized to the dissolved activity of the same
protease without the A215X substitution (−A215X).

| SEQ | | | | | | |
|---|---|---|---|---|---|---|
| ID | | | | | | |
| NO: | −A215X | +A215K | +A215Q | +A215N | +A215T | +A215S |
| 1 | 1.0 | 3.2 | — | — | — | — |
| 3 | 1.0 | 5.6 | — | — | — | — |
| 4 | 1.0 | 5.3 | — | — | — | — |
| 5 | 1.0 | 2.9 | — | — | — | — |
| 6 | 1.0 | 15.6 | 5.1 | 2.4 | 1.1 | 1.0 |

Table 2 shows the dissolved protease activity after 60 min of the A215X variants. As can be seen, the A215K substitution provided a 1.1 to 6.0-fold increase in dissolved protease activity for all five proteases tested. The substitutions A215Q, A215N, A215T, and A215S provided a 1.6 to 4.3-fold increase in crystal solubility. Although A215S substitution did not affect the degree of protease crystal formation (cf. Table 1), this substitution provided a 1.7-fold increase in dissolved activity after 60 min. Hence, these data indicate that the solubility of protease crystals is increased by introduction of A215X substitutions.

TABLE 2
Dissolved activity after 60 min of A215X variants. The dissolved activity
is given as a fold increase, normalized to the dissolved activity
of the same protease without the A215X substitution (−A215X).

| SEQ | | | | | | |
|---|---|---|---|---|---|---|
| ID | | | | | | |
| NO: | −A215X | +A215K | +A215Q | +A215N | +A215T | +A215S |
| 1 | 1.0 | 1.1 | — | — | — | — |
| 3 | 1.0 | 4.4 | — | — | — | — |
| 4 | 1.0 | 6.0 | — | — | — | — |
| 5 | 1.0 | 1.5 | — | — | — | — |
| 6 | 1.0 | 4.8 | 3.1 | 4.3 | 1.6 | 1.7 |

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A protease variant of a parent protease, wherein the variant has a sequence identity of at least at least 80%, but less than 100%, to SEQ ID NO:1;
wherein the variant comprises a first substitution selected from the group consisting of X215K, X215R, X215Q, X125N, X215S, and X215T;
wherein the variant comprises at least three further alterations, preferably substitutions, selected from the group consisting of X3T (e.g., S3T), X4I (e.g., V4I), X9E (e.g., S9E), I35ID, X43R (e.g., N43R), X76D (e.g., N76D), X99D (e.g., S99D, X99F (e.g., S99F), X101E (e.g., S101E), X101L (e.g., S101L), X103A (e.g., S103A), X103T (e.g., S103T), X104I (e.g., V104I), X120D (e.g., H120D), X160S (e.g., G160S), X195E (e.g., G195E), X205I (e.g., V205I), X206L (e.g., Q206L), X209W (e.g., Y209W), X235L (e.g., K235L), X259D (e.g., S259D), X261W (e.g., N261W), and X262E (e.g., L262E);
wherein the variant has protease activity; and
wherein position numbers are based on the numbering of SEQ ID NO:2.

2. The protease variant according to claim 1, wherein the first substitution is selected from the group consisting of X215K, X215Q, X125N, X215S, and X215T; preferably the first substitution is selected from the group consisting of X215K, X215Q, X125N, and X215T.

3. The protease variant according to claim 1, wherein the first substitution is selected from the group consisting of A215K, A215R, A215Q, A215N, A215S, and A215T; preferably the first substitution is selected from the group consisting of A215K, A215Q, A215N, A215S, and A215T; most preferably the first substitution is selected from the group consisting of A215K, A215Q, A215N, and A215T.

4. The protease variant according to claim 1, wherein the at least three further alterations, preferably substitutions, are selected from the group consisting of S3T, V4I, S9E, I35ID, N43R, N76D, S99D, S99F, S101E, S101L, S103A, S103T, V104I, H120D, G160S, G195E, V205I, Q206L, Y209W, K235L, S259D, N261W, and L262E.

5. The protease variant according to claim 4, wherein the at least three further alterations, preferably substitutions, are selected from one of the groups consisting of:
a) S3T, V4I, S99D, S101E, S103A, G160S, and V205I;
b) I35ID, N76D, H120D, G195E, K235L;
c) S9E, N43R, N76D, S99F, S101L, S103T, V104I, V205I, Q206L, Y209W, S259D, N261W, and L262E; and
d) S9E, N43R, N76D, V205I, Q206L, Y209W, S259D, N261W, and L262E.

6. The protease variant according to claim 1,
which has improved solubility compared to the parent protease; preferably the solubility is improved by at least 4%, e.g., at least 10%, at least 20%, at least 30%, at least 40%, at least 50%, at least 60%, at least 70%, at least 80%, at least 90%, at least 100%, at least 125%, at least 150%, at least 175%, at least 200%, at least 250%, at least 300%, at least 400%, at least 500%, or more, compared to the parent protease

7. The protease variant of claim 6, wherein the parent protease is an otherwise identical protease without the first substitution selected from the group consisting of X215K, X215R, X215Q, X125N, X215S, and X215T and without the at least three further alterations, preferably substitutions, selected from the group consisting of X3T (e.g., S3T), X4I (e.g., V4I), X9E (e.g., S9E), I35ID, X43R (e.g., N43R), X76D (e.g., N76D), X99D (e.g., S99D, X99F (e.g., S99F), X101E (e.g., S101E), X101L (e.g., S101L), X103A (e.g., S103A), X103T (e.g., S103T), X104I (e.g., V104I), X120D (e.g., H120D), X160S (e.g., G160S), X195E (e.g., G195E), X205I (e.g., V205I), X206L (e.g., Q206L), X209W (e.g., Y209W), X235L (e.g., K235L), X259D (e.g., S259D), X261W (e.g., N261W), and X262E (e.g., L262E).

8. The protease variant according to claim 6, wherein the variant has improved solubility compared to SEQ ID NO:1, SEQ ID NO:3, SEQ ID NO:4, SEQ ID NO:5, and/or SEQ ID NO:6.

9. The protease variant according to claim 6, wherein the protease variant has improved solubility at 10-30° C., preferably at 15-25° C., most preferably at 20° C.

10. The protease variant according to claim 6, wherein the protease variant has improved solubility at pH 3-9, preferably at pH 4-8, more preferably at pH 4-6, even more preferably at pH 4-5, most preferably at pH 4.5.

11. The protease variant according to claim 6, wherein the protease variant has improved solubility at 15-25° C. and pH 4-6, preferably at 20° C. and pH 4-5.

12. The protease variant according to claim 6, wherein improved solubility is determined as decreased protease crystal formation and/or increased protease crystal solubility according to Example 1.

13. The protease variant according to claim 1, which has on par or improved protease activity; preferably the protease activity is at least 100%, e.g., at least 101%, at least 102%, at least 103%, at least 104%, at least 105%, at least 110%, at least 120%, at least 130%, at least 140%, at least 150%, at least 175%, at least 200%, at least 250%, at least 300%, at least 400%, at least 500%, compared to the parent protease.

14. The protease variant according to claim 13, wherein the parent protease is an otherwise identical protease without the first substitution selected from the group consisting of X215K, X215R, X215Q, X125N, X215S, and X215T and without the at least three further alterations, preferably substitutions, selected from the group consisting of X3T (e.g., S3T), X4I (e.g., V4I), X9E (e.g., S9E), I35ID, X43R (e.g., N43R), X76D (e.g., N76D), X99D (e.g., S99D, X99F (e.g., S99F), X101E (e.g., S101E), X101L (e.g., S101L), X103A (e.g., S103A), X103T (e.g., S103T), X104I (e.g., V104I), X120D (e.g., H120D), X160S (e.g., G160S), X195E (e.g., G195E), X205I (e.g., V205I), X206L (e.g., Q206L), X209W (e.g., Y209W), X235L (e.g., K235L), X259D (e.g., S259D), X261W (e.g., N261W), and X262E (e.g., L262E).

15. The protease variant according to claim 13, wherein the variant has on par or improved protease activity compared to SEQ ID NO:1, SEQ ID NO:3, SEQ ID NO:4, SEQ ID NO:5, and/or SEQ ID NO:6.

16. A polynucleotide encoding a protease variant according to claim 1.

17. A nucleic acid construct or expression vector comprising a polynucleotide of claim 16.

18. A host cell expressing a protease variant according to claim 1.

19. A method for obtaining a protease variant according to claim 1, the method comprising:
(a) introducing into a parent protease a first substitution selected from the group consisting of X215K, X215R, X215Q, X125N, X215S, and X215T; and introducing at least three further alterations, preferably substitutions, selected from the group consisting of X3T (e.g., S3T), X4I (e.g., V4I), X9E (e.g., S9E), I35ID, X43R (e.g., N43R), X76D (e.g., N76D), X99D (e.g., S99D, X99F (e.g., S99F), X101E (e.g., S101E), X101L (e.g., S101L), X103A (e.g., S103A), X103T (e.g., S103T), X104I (e.g., V104I), X120D (e.g., H120D), X160S (e.g., G160S), X195E (e.g., G195E), X205I (e.g., V205I), X206L (e.g., Q206L), X209W (e.g., Y209W), X235L (e.g., K235L), X259D (e.g., S259D), X261W (e.g., N261W), and X262E (e.g., L262E); wherein the variant has protease activity; and
(b) recovering the variant.

20. A detergent composition comprising a protease variant according to claim 1 and one or more detergent components.

21. The detergent composition according to claim 20, wherein the composition is in the form of a bar, a homogenous tablet, a tablet having two or more layers, a pouch having one or more compartments, a regular or compact powder, a granule, a paste, a gel, or a regular, compact or concentrated liquid.

22. The detergent composition according to claim 20, wherein the composition is in the form of a liquid.

23. (canceled)

24. A method of cleaning laundry or a hard surface, the method comprising contacting the laundry or hard surface with a protease variant according to claim 1.
